(19) United States
(12) Patent Application Publication
(10) Pub. No.: US 2006/0106262 A1
(43) Pub. Date: May 18, 2006

(54) Electrooptic chromophores with large optical birefringence for applications at high speed and short wavelengths

(75) Inventors: Vincent D. McGinniss, Sunbury, OH (US); Steven M. Risser, Reynoldsburg, OH (US); Kevin B. Spahr, Worthington, OH (US)

Correspondence Address: Jerry K. Mueller, Jr.;Mueller and Smith, LPA, 7700 Rivers Edge Drive, Columbus, OH, 43235, US

(73) Assignee: Optimer Photonics, Inc.

(21) Appl. No.: 11/281,082

(22) Filed: Nov. 17, 2005

Related U.S. Application Data
(60) Provisional application No. 60/632,052, filed on Dec. 1, 2004.
(60) Provisional application No. 60/629,160, filed on Nov. 18, 2004.

(51) Int. Cl. C07C 17/42

(52) U.S. Cl. 570/113

(57) ABSTRACT

Disclosed is a series of materials, which exhibit large birefringence under the influence of an applied electric field. These materials are capable of switching this large birefringence with a characteristic time on the order of 1 microsecond or less. In addition, these materials have good optical loss at this wavelength, and are stable under irradiation. These materials are suitable for fabrication of optical devices such a variable optical attenuators, switches, and modulators that respond in these time frames or slower. These materials are also suitable for use across a wide range of wavelengths. As a second component of this invention, some of these novel materials exhibit these desired optical properties (large birefringence, low loss, stability under illumination) at wavelengths as short as about 400 nm. These materials are suitable for fabrication of optical devices operating at or about 405 nm, where conventional EO materials strongly absorb and/or quickly degrade.

### CROSS-REFERENCE TO RELATED APPLICATIONS

This application claims benefit of provisional applications Ser. Nos. 60/629,160 filed Nov. 18, 2004, entitled “Materials for use in high-speed optical modulators operating at or near 405 NM wavelengths”, and 60/632,052 filed Dec. 1, 2004 entitled “Novel materials with large birefringence and fast response”.

### FIELD OF THE INVENTION

The present invention relates in general to nonlinear optically active molecules and, more particularly to small organic chromophores having useful optical properties.

### BACKGROUND OF THE INVENTION

Electrooptic (EO) materials commonly are composed of a host polymer with a guest chromophore, included at about 5 to about 40 wt-% (weight percent). Most EO materials operate through the linear electrooptic effect (Pockels effect), in which the chromophores are aligned (poled) by an external electric field prior to operation of the device. To be an effective EO material, the chromophore must satisfy several requirements. It must possess a large dipole moment (to aid in chromophore alignment), it must possess a large nonlinearity along the dipole moment, it must be stable under continuous exposure at the operational wavelength, and it must be compatible with the host polymer.

Much current research has centered on the development of chromophores with larger EO coefficients, suitable for application at infrared wavelengths (850 nm, 1300 nm, 1550 nm, etc.). This has been achieved by extending the conjugation of the chromophore and incorporating stronger acceptor and donor moieties. As such, the absorption maximum of these newly developed chromophores has been further and further red-shifted to where the chromophores absorb in the near-infrared region. However, this increase in acceptor strength also leads to aggregation of the chromophore molecules in most polymers.

A second pressing issue for conventional (poled) EO materials is the stability of the poling. As the materials age, the chromophore molecules relax from the aligned configuration, leading to a reduction in the EO response. The recent research has concentrated on means of preventing chromophore relaxation from the aligned position, either through use of higher Tg polymers, attaching the chromophore as a sidechain, or crosslinking the polymer. Thus conventional materials are concerned with preventing chromophore reorientation on the timescale of years, while the present materials show chromophore motion on timescales smaller than a microsecond.

In addition to having large optical nonlinearities, chromophores typically have large anisotropy in their linear optical properties, Δα. This large optical anisotropy evidences itself in a large birefringence when the chromophores are oriented along a common direction (the direction of the electric field). This large birefringence is commonly used in photorefractive (PR) materials, where a photo-induced electric field is used to induce a response in a chromophore/polymer material at a temperature above the glass transition temperature of the material. The total response of the PR material has both an electronic and an orientational component. The relative magnitude of these two components is evaluated by examining the response of the material to high and low frequency applied electric fields. At low frequency, the chromophore rotates in conjunction with the applied field, leading to larger birefringence, while at high frequency, the chromophore is frozen in a given orientation, and responds only with the electronic component, leading to smaller birefringence. The ratio of these components can range from more than 30 for small chromophores to as low as 10 for large chromophores.

Several recent studies of PR materials (K. G. Jesperson, et. al., J. Opt. Soc. Am. B 20, 2179-2188 (2003); D. Van Steenwinckel, et. al., J. Chem. Phys. 112, 11030-11037, (2000); K. Hoechstetter, et. al., J. Chem. Phys. 110, 4944-4951 (1999); J. A. Herlocker, et. al., Appl. Phys. Lett. 74, 2253-2255 (1999); L. Mager, et. al., Appl. Phys. Lett. 71, 2248-2250 (1997); B. Swedek, et. al., J. Appl. Phys. 82, 5923-5925, (1997); B. Kippelen, et. al., Appl. Phys. Lett. 68, 1748-1750 (1996)) have examined novel materials with high PR performance. These materials were examined at both low and high frequency to determine the relative contribution of the orientational and electronic components of the birefringence. In all these cases, the studies showed that a 1 kHz electric field was suitable for the high frequency measurement, that is, in a polymer matrix above the glass transition temperature, the chromophore molecules could not significantly reorient in response to a 1 KHz electric field. This result then sets the response time for devices fabricated with these materials as being longer than 1 millisecond if using both the electronic plus orientational response.

This invention is focused on development of materials that are capable of displaying substantial orientational birefringence in response to much higher frequency electric fields, up to and surpassing 1 MHz in frequency. In addition, these materials also must meet additional requirements to be suitable for application in optical devices, such as having low absorption loss at the operational wavelength, being stable under extended illumination at the operational wavelength and at the operational temperature, stability under voltage for extended periods, and compatibility with the substrate. We will show there is a range of chromophores and polymers that can meet these requirements.

A second focus of this invention is on development of materials that work at the much shorter wavelength region near 405 nm. To be acceptable for this application, these materials must have low loss at this wavelength, must possess large EO coefficients, must be stable for extended periods under 405 nm illumination, and must be compatible with the host polymers. We will show there are a range of chromophores that can meet these requirements. However, because the chromophores are small in size, the can not be poled like conventional chromophores. Instead, the EO response is obtained by applying a continual bias to the EO material. Because the EO response is due to the presence of both the DC bias field and the modulating field, the response of the material is more properly described as a Kerr response rather than a Pockels response, as with conventional EO materials.

### SUMMARY OF THE INVENTION

Disclosed is a series of materials, which exhibit large birefringence under the influence of an applied electric field. These materials are capable of switching this large birefringence with a characteristic time on the order of 1 microsecond or less. In addition, these materials have good optical loss at this wavelength, and are stable under irradiation. These materials are suitable for fabrication of optical devices such a variable optical attenuators, switches, and modulators that respond in these time frames or slower. These materials are also suitable for use across a wide range of wavelengths.

As a second component of this invention, some of these novel materials exhibit these desired optical properties (large birefringence, low loss, stability under illumination) at wavelengths as short as about 400 nm. These materials are suitable for fabrication of optical devices operating at or about 405 nm, where conventional EO materials strongly absorb and/or quickly degrade.

### BRIEF DESCRIPTION OF THE DRAWINGS

FIG. 1 is a schematic of the sample used in the EO cell, where the light is polarized 45° out of the plane of the paper;

FIG. 2 is a schematic of Mach-Zehnder device used in this invention;

FIG. 3 is an EO response of an typical large EO chromophore, VC8, in CP087;

FIG. 4 is an EO response of an typical large EO chromophore, VC8, in CP087;

FIG. 5 is an EO response of a prototypical fast-response EO chromophore, 4A2TFMBN, in CP087;

FIG. 6 is an EO response of a prototypical fast-response EO chromophore, 4DMABN, in PMMA;

FIG. 7 is an EO response of a prototypical fast-response EO chromophore, 4DMABN, in CP087;

FIG. 8 is an EO response of a prototypical fast-response EO chromophore, 4DMABN, in PMMA;

FIG. 9 is an EO response of a prototypical fast-response EO chromophore, 4DMABN, in a highly fluorinated polymer;

FIG. 10 is n DC Vπ and RF Vπ for Yankees1 device at 480 MHz;

FIG. 11 graphically displaces the ratio of RF Vπ and DC Vπ for Yankees1 device at 480 MHz;

FIG. 12 graphically displays the response of the Allspice1 device to applied voltage;

FIG. 13 graphically displays the response of the 178-084-33#3 device to applied voltage;

FIG. 14 graphically displays the RF response of the 178-084-3 #3 device at 480 MHz;

FIG. 15 graphically displays the ratio of RF Vπ and DC Vπ for 178-084-33 #3 device at 480 MHz;

FIG. 16 graphically displays the frequency dependent response for Yankees1 and 178-084-33#3 devices;

FIG. 17 graphically displays the response of 178-084-33#3 to fast voltage step;

FIG. 18 graphically displays the EO response of 4DMABN in CP087;

FIG. 19 graphically displays the EO response of Odyssey 3 device to a step voltage; and

FIG. 20 graphically displays the EO response of Odyssey 3 device to a step voltage.

These drawings will be described in greater detail below.

### DETAILED DESCRIPTION OF THE INVENTION

In one aspect, the present invention provides an organic chromophore. The chromophore is an optically active compound that possesses large optical anisotropy and optionally large optical nonlinearities.

The chromophores of the present invention are characterized as having large optical anisotropy; large dipole moments; chemical, thermal, electrochemical, and photochemical stability; low absorption at operating wavelengths (e.g., about 1.3 and about 1.55 μm, about 405 nm and about 633 nm); suitable solubility in solvents suitable for the host polymers detailed below; compatibility with host polymer; and low volatility.

Standard EO Chromophores

Polymer-based EO materials contain chromophores, whether physically blended into the polymer, or covalently attached to the polymer as a sidechain, crosslinking, or backbone group. To exhibit a suitable EO response, the chromophore must satisfy several requirements. It must have an extended conjugated aromatic or conjugated core. It must possess a strong electron donating group at one end, and one or more strong electron accepting groups at the opposite end. This placement leads to a large molecular dipole moment. The chromophore also should not strongly absorb light at the operational wavelength of the material. The molecular quantities most important in determining the suitability of a chromophore are 1) the molecular dipole moment (μ), 2) the molecular first hyperpolarizability (β), and 3) the absorption maximum (λₘₐₓ). Materials which exhibit large orientational birefringence also require a large optical anisotropy, Δα. In most chromophores the large optical axis lies close to the axis along which the dipole moment is oriented.

Pockels Effect

The Pockels effect can only be exhibited by materials that lack a center of symmetry. For electrooptic polymers (or polymer/chromophore blends), which are isotropic, the intrinsic symmetry is broken by electric field poling. In this situation, the polymer is heated above its glass transition temperature. A strong electric field then is applied to the material. The interaction between the field and the dipole moment of the chromophore causes the chromophores (or optically active polymer components) to align along the field. The polymer then is cooled to below its glass transition temperature with the field still applied, causing the ordering to be frozen in, creating a uniaxial material. In poled polymeric materials, the Pockels coefficient r₃₃, is proportional to the product of the dipole moment, first hyperpolarizability, and poling field. A primary concern with materials using the Pockels effect is the rate at which the poling decays. Because the oriented (poled) material is not in a thermodynamic equilibrium state, the ordering will relax over time, leading to a reduced EO response. In guest-host materials, the rate of this decay depends on the size of the chromophore and the temperature difference between the testing temperature and the glass transition temperature of the polymer.

Kerr Effect

In the Kerr effect, the material's optical response is proportional to the square of the applied electric field. The electric field induces optical anisotropy either through molecular reorientation or alteration of the electronic structure of the medium. In the static field limit [C. J. F. Bottcher and P. Bordewijk, Theory of Electric Polarization, Elsevier Scientific Pub., 1973; W. T. Coffey and S. G. McGoldrick. “Inertial effects in the theory of dielectric and Kerr effect relaxation of an assembly of non-interacting polar molecules in strong alternating fields”. Chemical Physics, Vol. 120, pp. 1-35, (1988)], the Kerr response is:

$$[ K ] = \frac{2 π {N}_{A}}{405} [ \frac{3}{kT} ( {α}^{τ} : {α}^{τ} - 3 αα ) + 2 {I}^{τ} : {γ}^{τ} : {I}^{τ} + \frac{3}{{k}^{2} {T}^{2}} ( {α}^{τ} : {μ}^{ρ} {μ}^{ρ} - {αμ}^{2} ) + \frac{4}{kT} {μ}^{ρ} \cdot {β}^{τ} : {I}^{τ} ]$$

where μ, α, β, and γ are the molecular dipole moment, polarizability, first and second hyperpolarizabilities, respectively. The first two terms are associated with redistribution of the electronic structure of the molecules, while the third and fourth terms involve reorientation of the chromophores. For purely optical fields, only the first and second terms contribute. For chromophores with large dipole moment, the fourth term dominates the Kerr response when large DC fields are applied. In the case of an AC field combined with a DC field, the contribution of the third term decreases as the frequency of the applied AC field increases. The prior art has shown that for chromophore/polymer blends, use of an AC frequency of 1 kHZ or higher effectively eliminates the contribution of the orientational components of the Kerr effect. One of the components of this invention is to demonstrate chromophore/polymer blends where the orientational component of the Kerr effect is substantial at AC frequencies far in excess of 1 MHz.
EO Cell with a Birefringent Material

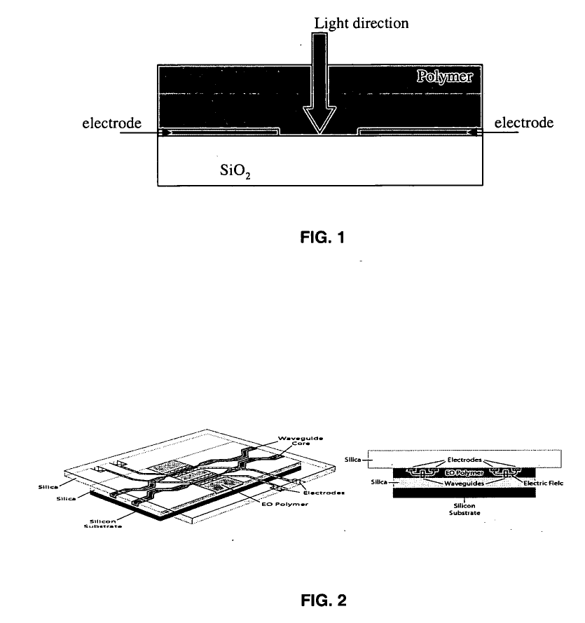

The electrooptic response of a chromophore can be measured in a polymer matrix using a transmission technique at telecommunication wavelength of 1.55 μm, or any other desired wavelength, such as 633 nm or 405 nm. A representative method for measuring the electro-optic coefficient is described in Nahata, et al., J. Opt. Soc. Am. B, 10, 1553 (1993). In this arrangement (shown in FIG. 1), two thin electrodes are laid down on a quartz slide, with a 20 μm gap between the electrodes. The polymer is applied to cover the two electrodes and fill the gap between them, and then is dried to remove all the solvent. Typical thickness of the polymer film ranges from about 10 to about 30 μm, and is measured for each individual cell.

To measure the EO effect in polymers, the polymer is heated (if desired), and both an AC and DC voltage is applied to the electrodes. For all EO measurements reported here, the AC frequency was 1 kHz, and the AC voltage was 200V peak-to-peak. The resulting electric field is oriented across the gap. A laser beam then is passed through the gap between the electrodes, with the beam polarized 45° to the direction of the gap. The refractive index for light polarized in the direction of the electric field is altered by the field, giving rise to a phase shift for this polarization. Light polarized orthogonal to the field has a smaller phase shift of opposite sign due to the applied voltage. Recombining the two polarizations then gives rise to a change in the transmitted power directly related to the change in the phase of the one polarization. Measurement of the transmitted power at the frequency of the AC voltage then gives a change in the phase proportional to the product of the AC and DC voltages. Note, that this analysis is simplified if the DC voltage is larger than the AC voltage. By varying both the temperature and the applied DC voltage, it is possible to determine the fraction of the material EO response due to either the Kerr or Pockels effect.

General Synthetic Materials and Methods

In the following examples, all analytical grade reagents and solvents were purchased from commercial sources including SigmaAldrich and VWR Scientific Products. The starting materials and solvents were used without further purification, unless otherwise noted. When dry reaction conditions are indicated, the solvents used are Aldrich Sure Seal grade; transferred via dry syringe or cannula. All reactions involving moisture or oxygen sensitive materials were performed in flame-dried glassware under a positive pressure of argon. Silica gel used for column chromatography was obtained from Aldrich with a mesh of 70-230 ASTM. Thin layer chromatography was done using EMS Science Silicagel 60 F₂₅₄ plates. All proton and carbon nuclear magnetic resonance spectra were recorded on a Bruker 600 MHz FTNMR spectrometer using CDCl₃ and tetramethylsilane as an internal reference. Melting points were obtained using a Fisher-Johns melting point apparatus and are reported uncorrected. Infrared spectra were obtained using a Perkin Elmer Spectrum RX FTIR. Visible spectra were obtained using a Shimadzu UV160U, using CHCl₃ as the solvent, and a nominal chromophore concentration of 1×10⁻⁵ on a weight basis.

Calculation of Molecular Properties

The molecular dipole moments (μ) and first hyperpolarizabilities (β) were calculated using ab initio electronic structure methods as implemented in JAGUAR™ (Jaguar 4.0, Schrodinger Inc., Portland, Oreg., 1991-2000). Ab initio methods have been shown to provide accurate descriptions of the dipole moments in small organic molecules. All the chromophore geometries were optimized using DFT methods with the B3LYP functional and the 6/31G* basis set. These resulting geometries were used for the calculation of the dipole moment and hyperpolarizability, using the CPHF method with a 6-31++G** basis.

The chromophore figure-of-merit (FOM) for the Pockels effect is determined by the formula:
PFOM=μβ/(molecular weight).
This PFOM, which depends on the first molecular hyperpolarizability, measures the response of a molecule to high-frequency signals, when in an oriented or poled state. When μ and β are calculated from electronic structure methods, the PFOM is the value at infinite wavelength, or PFOMₒ. The PFOM is an approximate measure of the effectiveness of a chromophore, with a higher PFOM corresponding to larger optical nonlinearities. Ideally, a chromophore would have a high PFOM, while being soluble in the polymers of interest. In reality, increasing the chromophore PFOM typically reduces the chromophore solubility and increases its tendency to aggregate, reducing the measurable PEO response of the material. The best chromophores are then those that have large PFOM values while still remaining active in the polymers of interest.

The magnitude electrooptic response of a chromophore depends not only on the properties of the chromophore, but also the wavelength at which the response is measured. This variation in the response, or dispersion, can be directly related to difference between the absorption maximum of the chromophore and the measurement wavelength by the following relation (Ph. Pretre, et al., J. Opt. Soc. Am. B, 15, 359-368 (1998)).

$$PFOM ( λ ) = {PFOM}_{o} \frac{( 3 - {λ}_{max}^{2} / {λ}^{2} )}{3 {( 1 - {λ}_{max}^{2} / {λ}^{2} )}^{2}}$$

This relation allows for determination of the response at one wavelength when a measurement has been taken at a second wavelength.
Host Polymers

The EO response of the chromophores was evaluated in one or more of the following polymers. Except where noted, the polymers were synthesized using standard procedures. Polycarbonate (PCARB) was purchased from Aldrich (˜26 k MW) and used as supplied. Polymethyl methacrylate (PMMA) was purchased from Polysciences (˜25 k MW) and used as supplied. The refractive index of this polymer at 1550 nm was measured to be n=1.477. Cellulose acetate butyrate (CAB) was purchased from Aldrich and used as supplied. The refractive index of this polymer at 1550 nm was measured to be n=1.4558. Norland UV epoxy 61 (NOR61), was purchased and used as supplied.

TABLE 1
Composition of polymers used in this invention
(Composition is based on mol-% of the starting monomers)

| | | Refractive |
|---|---|---|
| | | Index at |
| Name | Composition | 1550 nm |
| CP029 | 50% TFEMA, 40% TFPMA, 10% PEGMEMA | 1.4284 |
| CP050 | 15% HFIPMA, 85% MMA | 1.4524 |
| CP051 | 50% TFEMA, 50% MMA | 1.4384 |
| CP061 | 85% DFEMA, 15% HEMA | 1.451 |
| CP066 | 35% TFEMA, 55% DFEMA, 10% HEMA | 1.4371 |
| CP073 | 20% TFEMA, 65% TFPMA, 15% MAA | 1.4264 |
| CP076 | 40% TFEMA, 35% TFPMA, 10% MMA, 15% | 1.4294 |
| | HEMA | |
| CP087 | 85% TFPMA, 15% HEMA | 1.4266 |
| CP089 | 50% TFPMA, 50% MMA | 1.4378 |
| CP091 | 75% TFPMA, 15% HEMA, 10% PFST | 1.4315 |
| CP093 | 75% TFPMA, 15% HEMA, 10% THFMA | 1.4317 |
| SPIKE | 14.4% TFEMA, 80.6% TFPMA, 5% HEMA | 1.4181 |

TFEMA = trifluoroethyl methacrylate
TFPMA = tetrafluoropropyl methacrylate
HEMA = hydroxyethyl methacrylate
MMA = methyl methacrylate
HFIPMA = hexafluoroisopropyl methacrylate
DFEMA = difluoroethyl methacrylate
PEGMEMA = polyethyleneglycolmonomethylethermethacrylate
MAA = methacrylic acid
PFST = pentfluorostyrene
THFMA = tetrahydrofurfuryl methacrylate

Functional Cladding of Waveguides

In a Mach-Zehnder device configuration, the change in the effective refractive index of one arm can be accomplished by either altering the refractive index of the waveguide material, or that of the cladding, or both. In the second case, we refer to this as a functional cladding. As the beam propagates down the arm of the functionally-clad MZI, a substantial fraction of the power in the beam is contained in the cladding material. By altering the cladding refractive index, it is then possible to alter the effective refractive index of the propagating beam.

Mach-Zehnder Interferometer

Some of the present examples use a Mach-Zehnder interferometer (MZI), with the material of this invention as an active cladding (FIG. 2). A Mach-Zehnder interferometric device works by splitting light into two equal beams, altering the relative phase of the two beams, and then re-combining them. The relative phase difference between the beams allow for selection of the output port for the light. In a Y-splitting MZI, there is only a single input port and output port. By altering the relative phase of the beams traveling down the two arms, the beams can constructively or destructively interfere, controlling the power output from the device. In an electrooptic polymer, as an electric field is applied, the refractive indices of the polymer change. An EO polymer device works by using the electrooptic effect in the polymer to create the phase change.

Film Quality

Films of the chromophores and polymer, at the specified chromophore weight percentage, were cast onto quartz blanks from dioxane solution. After drying, the films were judged for their optical clarity and homogeneity on a scale of 1-10, with 1 being a completely perfect film. Polymer/chromophore solutions were deemed to be of sufficient quality for further testing if the film scored higher than a 4 on this scale. A set of reference films was employed to ensure consistency in the film evaluation.

Slab Waveguides

Slab waveguides were fabricated and tested for guiding quality in the following manner. The desired chromophore/polymer blend was placed into solution at a fixed weight percent solids. The concentration of the chromophore in the polymer was set so the refractive index of the blend at the testing wavelength was higher than that of the fused silica substrate. This solution was then spin-coated onto a fused silica slide and dried, and the thickness of the resulting film measured. The viscosity of the solution was then adjusted and a solution coated onto a new substrate until a film of approximately 3 μm thickness was formed. Either 405 nm or 633 nm laser light was prism coupled into the EO material, and after traversing approximately a 1 inch path, the light was then prism coupled out of the waveguide. The intensity and shape of the output spot was then evaluated to determine the guiding and loss properties of the film.

Chromophores of This Invention

The chromophores of this invention have the following general structure

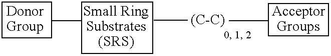

Specific donor group and acceptor group structures, and small ring substrates are presented in Tables 2, 3, and 4, along with relevant physical properties. These tables are not all-inclusive, but are designed to illustrate how various donor, acceptor or SRS groups would be categorized. Specific examples of chromophores of this invention are given in Table 5. More extensive listings of compounds used in this invention are given in Tables 6, 7, and 8:

TABLE 2
Donor Groups

| | Donor | Donor |
|---|---|---|
| | Wavelength | Speed |
| Examples of Donor Group | Parameter | Parameter |
| H— H₃C— | Weak | Light |
| Alkyl—N═ | Strong | Light-Heavy |
| Aromatic— | Strong | Medium-Heavy |
| R—O— | Weak | Light-Heavy |
| R—S— | Weak | Medium-Heavy |
| 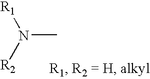 | Strong | Medium-Heavy |
| 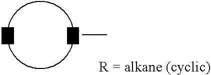 | Strong | Heavy |
| 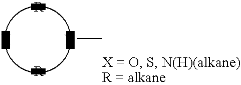 | Strong | Heavy |

TABLE 3
Acceptor Groups

| | Acceptor | |
|---|---|---|
| | Wavelength | Acceptor |
| Examples of Acceptor Groups | Parameter | Speed Parameter |
| —F | Weak | Light |
| —NO₂ | Medium | Light |
| —CN | Weak | Light |
| —CF₃ | Weak | Medium |
| —SO₂CF₃ | Strong | Medium |
| —SO₂CH₃ | Strong | Medium |
| 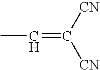 | Strong | Medium |
| 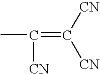 | Strong | Medium |
| 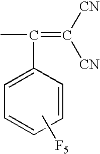 | Strong | Heavy, Branched |
| 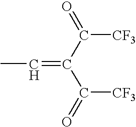 | Strong | Heavy, Branched |
| 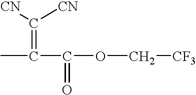 | Strong | Heavy, Branched |
| 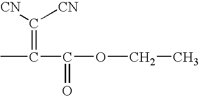 | Strong | Heavy, Branched |

TABLE 4
Small Ring Substrates (SRS)

| | Small |
|---|---|
| Examples of Small Ring Substrates | Ring Substrate Parameter |
| 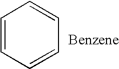 | Medium |
| 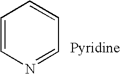 | Medium |
| 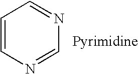 | Medium |
| 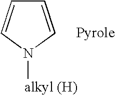 | Medium-Heavy |
| 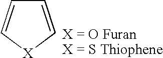 | Medium |
| 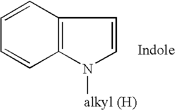 | Medium-Heavy |
| 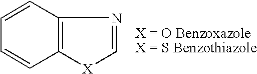 | Medium |
| 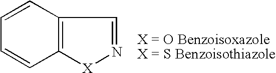 | Medium |
| 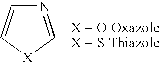 | Medium |
| 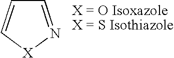 | Medium |
| 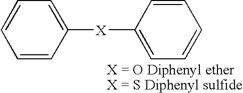 | Medium |
| 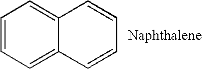 | Medium |
| 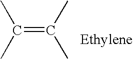 | Light |

The criterion for determining the value of the Donor Wavelength Parameter (DWP) is the measured, or predicted, value of the absorption maximum for the molecule formed according to the prescription above, where the Small Ring Substrate is a phenyl and the Acceptor Group is a nitro group, located para to the donor. A donor group with a weak DWP is one that leads to an absorption maximum of approximately 330 nm or less for this chromophore, while a donor group with a strong DWP is one that leads to an absorption maximum of more than approximately 330 nm for this chromophore.

The criterion for determining the value of the Acceptor Wavelength Parameter (AWP) is the measured, or predicted, value of the absorption maximum for the molecule formed according to the prescription above, where the Small Ring Substrate is a phenyl and the Donor Group is a dimethylamine group, located para to the acceptor. An acceptor group with a weak AWP is one that leads to an absorption maximum of approximately 340 nm or less for this chromophore, an acceptor group with a medium AWP is one that leads to an absorption maximum approximately between about 340 nm and about 400 nm for this chromophore, while an acceptor group with a strong AWP is one that leads to an absorption maximum approximately greater than about 400 nm for this chromophore.

The criteria for determining the value of the Donor Speed Parameter (DSP), the Acceptor Speed Parameter (ASP), and the Small Ring Substrate Parameter (SRSP) are based on the atomic mass or size of the group. A donor group with a weak DSP is one that has an atomic mass of less than approximately 40 amu, a donor group with a medium DSP has an atomic mass between approximately 40 amu and approximately 70 amu, while a donor group with a strong DSP is one that has an atomic mass of more than approximately 70 amu. An acceptor group with a weak ASP is one that has an atomic mass of less than approximately 50 amu, an acceptor group with a medium ASP has an atomic mass between approximately 50 amu and approximately 150 amu, while an acceptor group with a strong ASP is one that has an atomic mass of more than approximately 150 amu. A small ring substrate with a light Small Ring Substrate Parameter (SRSP) is a group that has less than approximately 5 non-hydrogen atoms, a small ring substrate with a medium Small Ring Substrate Parameter (SRSP) is a group that has between approximately 5 non-hydrogen atoms and 12 non-hydrogen atoms, while a small ring substrate with a heavy Small Ring Substrate Parameter (SRSP) is a group that has more than approximately 12 non-hydrogen atoms.

There is a simple process used to determine whether a given chromophore may be suitable for use at about 405 nm, or may provide fast response. The first criterion for a chromophore to have a useful response at about 405 nm is that its absorption must be small about at 405 nm. A primary indicator of this is the location of the chromophore's absorption maximum. For weak absorption at about 405 nm, the chromophore absorption maximum must be less than approximately 330 nm. An alternate method of determining whether a chromophore may have a useful response at about 405 nm is based on the strength of the donor and acceptor groups of the chromophore. Possible combinations include, inter alia:

| Donor | SRS | Acceptor |
|---|---|---|
| Weak | Light or Medium | Weak, Medium, or |
| | | combination |
| Strong | Light or Medium | One or more weak |
| Weak | Heavy | One or more weak |

Note that none of the strong acceptors can be used for chromophores that have useful response at about 405 nm.

There is a similar process to determine whether a chromophore may be capable of the fast response described in this invention. Although there are multiple factors that determine whether a chromophore/polymer blend has a useful fast EO response according to this invention, including details of the interaction between the polymer and chromophore, a primary criterion for determining whether a useful response may be possible is based on the size and shape of the chromophore. Chromophores that are too large or too bulky will not have a useful fast response. We have found that a chromophore with a molecular weight above approximately 350 amu will not have a fast response. An alternate method of determining whether a chromophore may have a fast response is outlined in the following selection criteria, based on the approximate size of the donor, SRS, and acceptor groups of the chromophore.

| Donor | SRS | Acceptor |
|---|---|---|
| Heavy | Light | Light |
| Light or Medium | Light | Light, Medium, or |
| | | Branched Medium |
| Light or Medium | Light or Medium | Light or Medium |

To better demonstrate these criteria, we now show how they can be used is specific examples. 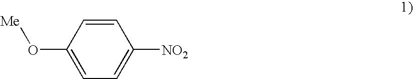
For 405 nm purposes, the molecule is composed of a weak donor, medium SRS, and medium acceptor. This is a predicted combination and the measured absorption maximum is 302 nm. For fast chromophore purposes, the donor is light, the SRS is medium, and the acceptor is light. This is one of the designated combinations, so this chromophore has a good probability of having a fast response. 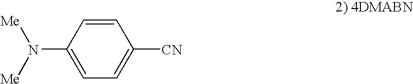
For 405 nm purposes, the molecule is composed of a strong donor, medium SRS, and weak acceptor. This is a predicted combination and the measured absorption maximum is 290 nm. For fast chromophore purposes, the donor is medium, the SRS is medium, and the acceptor is light. This is one of the designated combinations, and the fast response of this chromophore is documented herein 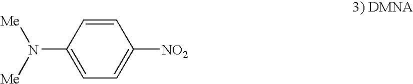
For 405 nm purposes, the molecule is composed of a strong donor, medium SRS, and medium acceptor. This is a combination that is not one of the acceptable combinations. The measured absorption maximum of this chromophore is 407 nm, which precludes its use as an EO chromophore at 405 nm. For fast chromophore purposes, the donor is medium, the SRS is medium, and the acceptor is light. This is one of the designated combinations, and the fast response of this chromophore is documented herein 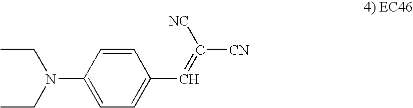
For 405 nm purposes, the molecule is composed of a strong donor, medium SRS, and medium acceptor. This is a combination that is not one of the acceptable combinations. The measured absorption maximum of this chromophore is 439 nm, which precludes its use as an EO chromophore at 405 nm. For fast chromophore purposes, the donor is medium, the SRS is medium, and the acceptor is medium. This is one of the designated combinations, and the fast response of this chromophore has been shown 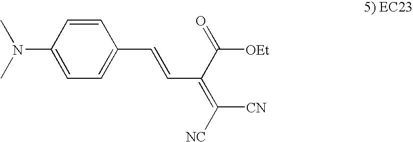
For 405 nm purposes, the molecule is composed of a strong donor, medium SRS, and strong acceptor. This is a combination that is not one of the acceptable combinations. The measured absorption maximum of this chromophore is 527 nm, which precludes its use as an EO chromophore at 405 nm. For fast chromophore purposes, the donor is medium, the SRS is medium, and the acceptor is branched medium. This is not one of the designated combinations, and the lack of a fast response of this chromophore is documented herein. 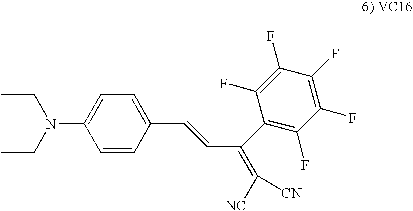
For 405 nm purposes, the molecule is composed of a strong donor, medium SRS, and strong acceptor. This is a combination that is not one of the acceptable combinations. The measured absorption maximum of this chromophore is 534 nm, which precludes its use as an EO chromophore at 405 nm. For fast chromophore purposes, the donor is medium, the SRS is medium, and the acceptor is heavy. This is not one of the designated combinations, and the lack of a fast response of this chromophore is expected.

Although this demonstrates the usefulness of these selection criteria, it needs to be stated that these are not foolproof. In particular, these criteria are useful for excluding chromophores but do not guarantee that a chromophore which meets these criteria will be useful.

TABLE 5
Specific Structures of this Invention

| Structure | Name | Melting Point | Molecular Weight |
|---|---|---|---|
| 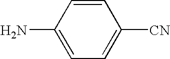 | p-cyanoanaline | 51-53 C. | 118.14 |
| 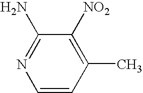 | 2-amino-3-nitro-4-methylpyridine | 139-141 C. | 153.14 |
| 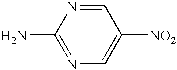 | 2-amino-5nitropyrimidine | 235-237 C. | 140.1 |
| 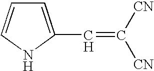 | 2-(1,1-dicyanoethylene)pyrrole | | 143 |
| 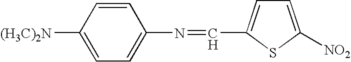 | 2-(p-dimethylaminophenyl-imino- methane)-5-nitrothiophene | | 275 |
| 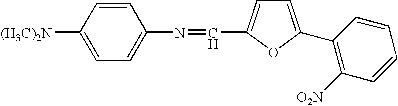 | 2-(p-dimethylaminophenyl-imino- methane)-5-(2-nitrophenyl)furan | | 335 |
| 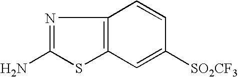 | 2-amino-6-(methylsulfonyl)benzo- thiazole | 223 C. | 228.29 |
| 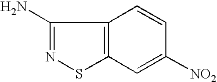 | 3-amino-5-nitrobenzothiazole | 250 C. | 195.2 |
| 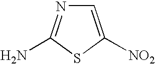 | 2-amino-5-nitrothiazole | 202 | 145.14 |
| 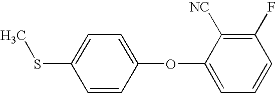 | 2-fluoro-6-[4-(methylthio)phen- oxy]benzonitrile | 68-71 C. | 259.2 |
| 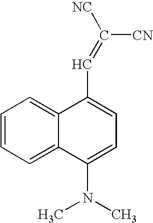 | Propanedinitrile,[[4-(di- methylamino)-1-naph- thalenyl]methylene] | | 247.30 |
| 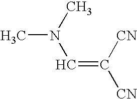 | 1-(dimethylamine)-2,2 dicyanoethylene | 84-85 C. | 121.14 |

TABLE 6
Other Benzonitriles

4-n-butylbenzonitrile
4-tert-butylbenzonitrile
3-chloro-5-fluoro-4-methoxybenzonitrile
2-chloro-5-methylbenzonitrile
2,4-difluoro-3-methoxybenzonitrile
4,5-dimethoxy-2-nitrobenzonitrile
2-dimethylamino-6-fluorobenzonitrile
2,3-dimethylbenzonitrile
2-ethoxybenzonitrile
2-ethylbenzonitrile
2-fluoro-6-(4-chlorophenylthio)benzonitrile
Phenyloxybenzonitrile
hydroxybenzonitriles
methoxybenzonitriles
alkylbenzonitriles

TABLE 7
Amino Benzonitriles

2-aminobenzonitrile
3-aminobenzonitrile
4-aminobenzonitrile
4-amino-3-chloro-5-methylbenzonitrile
4-amino-2,5-difluorobenzonitrile
2-amino-4,5-dimethoxybenzonitrile
4-amino-3-ethylbenzonitrile
2-amino-6-fluorobenzonitrile
5-amino-2-fluorobenzonitrile
Dicyanoaniline
4-{5-(4-amino-phenyl)-tetrazol-2-ylmethyl}-benzonitrile
4-amino-3-(trifluoromethoxy)benzonitrile

TABLE 8
Other Compounds

Amino-nitro-pyridines
Trifluoromethyl-nitrile-aniline
n-{2-nitro-4-(trifluoromethyl)phenyl}morpholine
n-{ibid}-piperazine
n-{ibid}-piperidine
n-{ibid}-pyrrolidine
Trifluoromethoxy aniline
Alkyl substituted amino-nitro-pyridines
Fluoro-nitro-anilines
Methoxy-nitrobenzenes
Aminophenyl trifluoromethyl sulfones
Amino-trifluoromethyl-benzimidazoles
Methyl-nitro-indoles
Nitro-phenyl imidazoles
3-amino-5-nitrobenzisothiazole
2-amino-5-nitropyrimidine
2-amino-6-nitrobenzothiazole
2-amino-5-nitrothiazole
2-amino-5-4-nitrophenylsulfonyl thiazole
4,4′ aminonitro-phenyl sulfide
Methoxy-amino-benzotrifluoride
Phenyloxy benzonitrile
1-dimethylamino-2-dicyanoethane
Aminobenzotrifluoride
cyanophenylsulfide

The following examples show how the present invention has been practiced, but should not be construed as limiting. In this application all units are in the metric system and all amounts and percentages are by weight, unless otherwise expressly indicated. Also, all citations referred herein are expressly incorporated herein by reference.

### EXAMPLES

### Example 1

### Properties of Small Chromophores

In this example, several prototypical small chromophores, along with their calculated (and measured if available) properties are presented. The values are given in Table 9. The values of μ and μβ for the experimental PFOM are taken from L.-T. Cheng, et. al., J. Phys. Chem. 95, pp. 10631 (1991). The calculated results were obtained using JAGUAR, as described above.

TABLE 9
Properties of Small Chromophores

| | | | | expt. | | calc. |
|---|---|---|---|---|---|---|
| Molecule | Abbreviation | MW | λ max | FOM | calc. μβ | FOMₒ |
| 2,5 bis-trifluoromethyl aniline | 2,5-BTFMA | 229.12 | | | 2.95 | 0.013 |
| 4 trifluoromethyl aniline | 4-TFMA | 161.13 | | | 7.88 | 0.049 |
| 3,5 bis-trifluoromethyl aniline | 3,5-BTFMA | 229.12 | | | 6.58 | 0.029 |
| nitrobenzene | NB | 123.11 | ˜260 nm | 0.063 | 3.78 | 0.031 |
| α,α,α trifluorotoluene | AAATFT | 146.11 | | | 3.77 | 0.026 |
| N,N, dimethyl 4 nitro analine | DMNA | 166.18 | 407 nm | 0.448 | 70.57 | 0.425 |
| 4-(dimethylamino)benzonitrile | 4DMABN | 146.19 | 290 nm* | 0.192 | 47.85 | 0.327 |
| 4-(dimethylamino)-2,2,2-trifluoroacetophenone | 4DMATFAP | 189.14 | 356 nm* | 0.312 | 76.08 | 0.402 |
| 3 methyl 4amine nitrobenzene | 3M4ANB | 152.15 | | 0.354 | 54.31 | 0.357 |
| 4 nitro aniline | 4NA | 138.13 | 365 nm** | 0.413 | 55.16 | 0.399 |
| 4-amino-4′-nitrophenyl sulfide | 4A4NPS | 246.29 | 350 nm | | 104.66 | 0.425 |
| 3-chloro-4nitro aniline | 3C4NA | 172.57 | | 0.232 | 47.27 | 0.274 |
| 4-amino-3-ethylbenzonitrile | 4A3EBN | 146.19 | | | 27.18 | 0.186 |
| 2 methyl, 3 trifluoromethyl aniline | 2M3TFMA | 175.15 | | | 1.95 | 0.011 |
| 2 fluoro, 4 trifluoromethyl aniline | 2F4TFMA | 179.12 | | | 7.09 | 0.040 |
| 2 trifluoromethyl, 4 nitro aniline | 4N2TFMA | 206.12 | | | 40.74 | 0.198 |
| 3 trifluoromethyl, 4 nitro aniline | 4N3TFMA | 206.12 | | | 48.88 | 0.237 |
| 4 aminophthalonitrile | 4APTN | 143.15 | 321.5 nm | | 47.54 | 0.332 |
| N-methyl-N-(hydroxyethyl) amino benzonitrile | 4HEMABN | 176.22 | 293.6 nm | | 47.99 | 0.272 |
| 4-amino-2-trifluoromethylbenzonitrile | 4A2TFMBN | 186.14 | 273.5 nm | | 31.82 | 0.171 |
| 3-fluoro-6-(4-methylphenoxy)benzonitrile | 3F6MPBN | 227.24 | | | 1.18 | 0.005 |
| 2-fluoro-6-[4-methylthio)phenoxy)benzonitrile | 2F6MPBN | 259.30 | | | 10.18 | 0.039 |
| (dimethylaminomethylene)malononitrile | DMAMMN | 121.14 | | | 2.03 | 0.017 |
| 3-amino-5-nitrobenzisothiazole | 3A5NBT | 195.20 | | | 12.14 | 0.062 |
| 2-amino-6-nitrobenzothiazole | 2A6NBT | 195.20 | | | 84.27 | 0.432 |
| 2-amino-5-nitropyrimidine | 2A5NP | 140.10 | | | 33.78 | 0.241 |
| 2-amino-4-methyl-5-nitropyridine | 2A4M5NP | 153.14 | | | 38.16 | 0.249 |
| 2-amino-5-(4-nitrophenylsulfonyl)thiazole | 2A5NPST | 285.30 | | | 30.38 | 0.106 |
| 3,5-dimethoxybenzonitrile | 35DMOBN | 163.18 | | | 10.19 | 0.062 |
| 4-amino-3-(trifluoromethyl)benzonitrile | 4A3BN | 186.14 | | | 21.36 | 0.115 |
| 4-amino-3-(trifluoromethoxy)benzonitrile | 4A3TFMBN | 202.14 | | | 32.46 | 0.161 |
| 4-aminobenzonitrile | 4ABN | 118.14 | | | 39.78 | 0.337 |
| 4-(diethylamino)benzonitrile | 4DEABN | 174.25 | | | 62.79 | 0.360 |
| N-(dinitrophenyl)-L-alanine methyl ester | DNPA | 269.21 | | | 12.62 | 0.047 |
| 2-methyl-4-nitroaniline | MNA | 152.15 | | | 58.10 | 0.382 |
| methylsulfonylacetone | MSA | 136.17 | | | 5.46 | 0.040 |

*measured in dioxane
**measured in acetone

### Example 2

### EO Properties of Standard Chromophore/Polymer Blend (178-086-02)

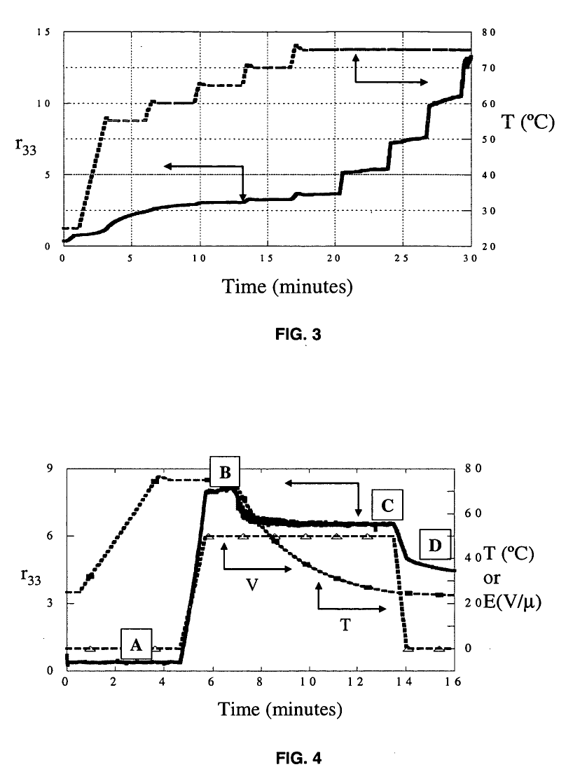

In this example we present the EO response of a standard OPI chromophore, VC8 (p-diethylamino-phenyl-hexa-1,2,7-triene,1-pentafluoro, 2,2-dicyanoethylene), in a partially fluorinated polymer, CP087 at 8.49 wt-%. The first EO trace, shown in FIG. 3, measures the response of the chromophore at 1 kHz to changes in temperature and voltage, using a 1550 nm laser as the optical source. The temperature and voltage profile used for this result will be referred to as Profile1 in the subsequent examples.

Profile1 begins with the EO cell held at room temperature. A DC bias of 25V/μm (500 V) is placed across the two electrodes in addition to the 200 V peak-to-peak AC voltage. The temperature is then quickly ramped to 55° C., held constant for approximately three minutes, then ramped to 60° C. This process continues until the sample reaches a temperature of 75° C. After approximately 3 minutes at this temperature and field, the temperature is now held constant and the field increased (around time of 20 minutes). The field is increased in 10V/μm increments (200 V), again holding the voltage constant for approximately 3 minutes between steps. The starting temperature of 55° C. for the material was chosen as the lowest temperature at which a response was obtained from the material. At temperatures below this, the response was indistinguishable from noise, independent of the applied bias voltage. The EO response of the material at each of these temperatures and voltages is summarized in Table 10.

TABLE 10
Summary of the EO response of an typical large EO chromophore

| Temperature | Bias field | EO |
|---|---|---|
| 55 C. | 25 V/μm | 1.8 pm/V |
| 60 C. | 25 V/μm | 2.75 pm/V |
| 65 C. | 25 V/μm | 3.05 pm/V |
| 70 C. | 25 V/μm | 3.26 pm/V |
| 75 C. | 25 V/μm | 3.62 pm/V |
| 75 C. | 35 V/μm | 5.25 pm/V |
| 75 C. | 45 V/μm | 7.3 pm/V |
| 75 C. | 55 V/μm | 10.1 pm/V |
| 75 C. | 65 V/μm | 13.2 pm/V |

The second EO trace, shown in FIG. 4, measures the response of the chromophore at 1 kHz in the poled, or aligned, state. The temperature and voltage profile used for this result will be referred to as Profile2 in the subsequent examples. Profile2 is similar to the steps one would use to pole a conventional EO material,

Profile2 begins with the EO cell held at room temperature with no bias field, but a 200 V peak-to-peak 1 kHz AC voltage applied across the electrodes. At a time of about 0.5 minutes the temperature begins ramping to 75° C. By time t=4 minutes, the temperature has stabilized at 75° C. At about t=5 minutes, a DC bias of 50V/μm is added to the electrodes. After approximately 2 minutes at temperature and voltage, the temperature is allowed to decrease, and by t=13.5 minutes, the EO material has returned to room temperature. At this point, the bias voltage is removed from the electrodes.

The EO response of the system has approximately four distinct points where it can be measured. The value of the response at each point is given in Table 11.
A. Initial Response. The response of the system before the application of DC bias voltage should be zero when measured at the frequency of the AC voltage.
B. “DCEO”. The EO response of the system to high voltage and temperature will be similar to that exhibited by the material during the first type of EO trace. Because the EO measurements are taken at an AC frequency of 1 kHz, this stage actually helps measure the ability of the chromophore to rotate at this angular frequency.
C. “RFEO”. The EO response of the system at high voltage and low temperature measures the purely electronic response of the chromophore, oriented by the applied DC bias, assuming room temperature is sufficiently low to prevent chromophore motion in response to the 1 kHz AC voltage. If the chromophore is able to reorient to some degree at room temperature in response to the AC voltage, the “RFEO” will be larger than the purely electronic response
D. “Poled EO”. The EO response of the system after the bias voltage and temperature have been removed. This response is the response commonly discussed in the literature when describing the EO response of Pockels materials. The rate at which this response decays after removal of the DC voltage provides information about the ability of the chromophore to maintain orientation at room temperature.

The combined response of Points B, C, and D allow for determination of the material response. A material where the “DCEO” is much larger than the “RFEO” means there is high chromophore mobility at elevated temperature. The “Poled EO” value then helps determine whether the “RFEO” is purely electronic (“Poled EO” close to the “RFEO” value), or still primarily orientational (“Poled EO” is much smaller than “RFEO”). A material where the “DCEO” and “RFEO” are almost the same, and the “Poled EO” is also large is a material where the “RFEO” is purely electronic and the small increase in the “DCEO” indicates the chromophore can not follow a 1 kHz AC voltage. A material where the “DCEO” and “RFEO” are almost the same, and the “Poled EO” is very small is a material where the “DCEO” and “RFEO” are both dominated by chromophore orientation, so at room temperature the chromophore is still very mobile in response to a 1 kHz AC voltage.

TABLE 11
Summary of the EO response of a typical large EO chromophore

| Point | EO Value |
|---|---|
| A | <0.4 pm/V |
| B | 8.0 pm/V |
| C | 6.5 pm/V |
| D | 4.5 pm/V |

The values in Table 11 fully support the standard view of the response of large chromophores in a polymer host. At Point A, the chromophores have not been exposed to a bias voltage, so there is no preferred orientation axis, meaning there is also no Pockels, or electronic response of the system. Any orientation of the chromophores in response to the purely AC voltage will appear to have a response at twice the AC frequency, so it will not be detected by the lock-in amplifier. At Point B, the chromophore molecules have been partially aligned by the bias voltage, and the polymer matrix has been softened, allowing the chromophore molecules to also slightly respond to the 1 kHz AC voltage. At Point C, the response is now purely electronic, as the chromophores in the frozen polymer matrix can not change orientation in response to the 1 kHz AC voltage. At Point D, the removal of the bias field leads to a rapid small relaxation of the chromophore molecules, as is well known in the literature. The EO response will continue to decrease with time as the chromophores continue to move towards an isotropic distribution. The presence of the large EO at point D also verifies the relative immobility of the chromophore molecules at room temperature in this host polymer and that the response at Point C is primarily electronic in nature.

The relative magnitude of the response at Point B and Point C provides much information about the ability of the chromophore to respond at 1 kHz. The birefringence induced at Point B contains both the electronic and orientational components, while that at Point C contains on the electronic component. In this material, at 1 kHz, the ratio of the responses is only ˜1.2. At very low frequency, this ratio has been shown in our labs to be approximately 10, consistent with published values for other chromophores (Sandalphon, et al., Appl. Opt. 35, 2346, (1996) reports a ratio of 17 for a diazo chromophore). This small ratio of the responses also verifies the assertion in the literature cited previously that 1 kHz is a sufficiently high frequency that the electronic response can be separated from the orientation response for typical EO chromophores. This ratio also supports the assertion that typical EO materials of a chromophore embedded in a host polymer matrix can not be used to fabricate optical devices requiring a reorientation response that respond in under a millisecond.

### Example 3

### EO Properties of Fast-Response EO Material (178-090-25)

In this example, we present the EO response of a prototypical fast-response chromophore, 4A2TFMBN (4-amino, 2-trifluoromethyl benzonitrile), in a partially fluorinated polymer, CP087 at 21.6 wt-%. The solution was also coated onto a prism for a refractive index measurement. The value of the index was measured to be 1.465 at room temperature and 1550 nm. The response of the chromophore at 1 kHz using EO Profile1 is shown in Table 12, using a 405 nm laser as the optical source. Here we see that the response is large at room temperature, which will complicate analysis of the results using EO Profile2.

TABLE 12
Summary of the EO response of a prototypical
fast-response EO chromophore

| Temperature | Bias field | EO |
|---|---|---|
| 25 C. | 25 V/μm | 12.0 pm/V |
| 29 C. | 25 V/μm | 13.9 pm/V |
| 29 C. | 35 V/μm | 18.2 pm/V |

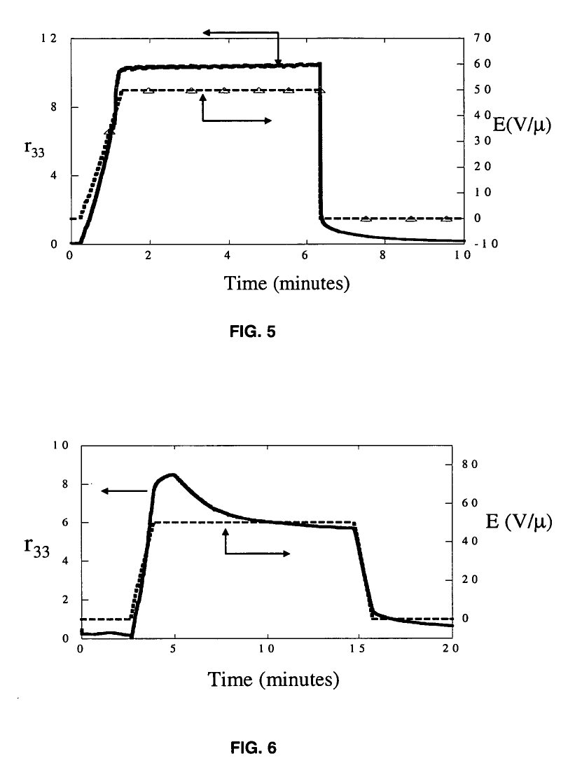

The EO response of this material is shown in FIG. 5. Because of the large response of the material at room temperature, the sample is never heated, but the bias voltage is just applied and removed. The results shown in this figure come from an EO cell that was exposed to the 405 nm source for approximately 65 hours over the course of a weekend. The response of the cell after the long-term exposure was the same as before the exposure, within experimental uncertainty, showing the response is stable at this wavelength.

There are a few important distinctions between these results and those of the typical chromophore shown in FIG. 4. The first is the large difference in the temperature needed to allow orientational response. The standard EO chromophore, VC8, required a temperature of at least 55° C. before the molecules could reorient under the influence of a DC field, while the 4A2TFMBN has high rotational mobility at room temperature. This is also evidenced by the incredibly rapid fall-off of the response for 4A2TFMBN. The removal of the bias field causes the EO to immediately drop by more than 90%, with a rapid decay of the residual EO.

### Example 4

### EO Properties of Small EO Material (178-093-06)

In this example we present the EO response of a small chromophore, 4N3TFMA (4-nitro, 3-trifluoromethyl analine), in a partially fluorinated polymer, CP087 at 20.0 wt-%. The response of the chromophore at 1 kHz using EO Profile1 is shown in Table 13, using a 1550 nm laser as the optical source. The 4N3TFMA chromophore shows much smaller response than 4DMABN, even though the chromophores are similar in size. The response of the material to Profile2 demonstrates the response in Table 13 is primarily orientational in nature.

TABLE 13
Summary of the EO response of a small EO chromophore.

| Temperature | Bias field | EO |
|---|---|---|
| 25 C. | 25 V/μm | 3.25 pm/V |
| 25 C. | 35 V/μm | 4.70 pm/V |
| 25 C. | 45 V/μm | 6.20 pm/V |
| 25 C. | 55 V/μm | 7.60 pm/V |
| 25 C. | 65 V/μm | 8.70 pm/V |
| 25 C. | 75 V/μm | 11.2 pm/V |

### Example 5

### EO Properties of Small EO Material (178-084-25)

In this example we present the EO response of a small chromophore, 4APTN (4 aminophthalonitrile), in PMMA at 20.2 wt-%. The response of the chromophore at 1 kHz using EO Profile1 is shown in Table 14, using a 405 nm laser as the optical source. Here again, we see there is a strong response at room temperature, even though the chromophore is placed in PMMA. Previous studies using PMMA as the host polymer for EO chromophores have shown that poling needs to take place at temperatures of at least 85° C. to have chromophore mobility, where we have shown chromophore mobility at least 60° C. below the standard accepted temperature.

TABLE 14
Summary of the EO response of a small EO chromophore

| Temperature | Bias field | EO |
|---|---|---|
| 25° C. | 25 V/μm | 2.9 pm/V |
| 29° C. | 25 V/μm | 3.54 pm/V |
| 33° C. | 25 V/μm | 3.87 pm/V |
| 37° C. | 25 V/μm | 4.5 pm/V |
| 41° C. | 25 V/μm | 4.9 pm/V |
| 41° C. | 35 V/μm | 8.6 pm/V |
| 41° C. | 45 V/μm | 11.8 pm/V |

The response of the chromophore at 1 kHz using EO Profile2 is shown in FIG. 6, using a 405 nm laser as the optical source.

### Example 6

### EO Properties of Small EO Material (178-090-21)

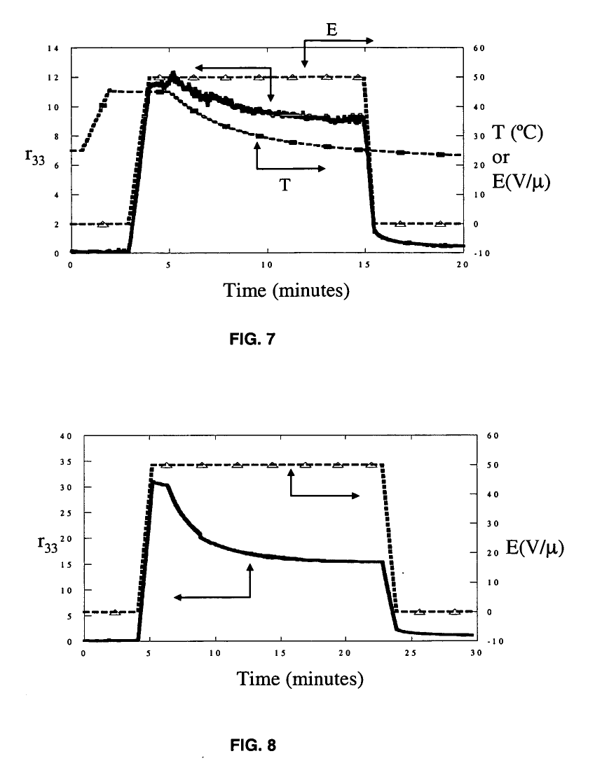

In this example we present the EO response of a small chromophore, 4DMABN (4 dimethylamino benzonitrile), in CP087 at 21.6 wt-%. The response of the chromophore at 1 kHz using EO Profile2 is shown in FIG. 7, using a 1550 nm laser as the optical source.

Here we see the chromophore is able to orient in conjunction with the 1 kHz AC voltage at room temperature, and that the EO drops off sharply when the bias field is removed from the system. The response of the system is stable. In a second test, the bias voltage was reapplied to the EO cell, with the EO value remaining constant over the next 30 minutes of testing, at which point the test was terminated.

The refractive index of this material was measured at 1550 nm, using standard procedures. The refractive index of the base polymer is 1.4266, and the refractive index of 21.6 wt-% 4DMABN in CP087 was measured to be 1.4613.

### Example 7

### EO Properties of Small EO Material (178-084-10)

In this example we present the EO response of small chromophore 4DMABN in PMMA at 21.7 wt-%. The solution also was coated onto a prism for a refractive index measurement. The value of the index was measured to be 1.5009 at room temperature and 1550 nm. The response of the chromophore at 1 kHz using EO Profile1 is shown in Table 15, using a 405 nm laser as the optical source. Here we see there is a strong response at room temperature, even though the chromophore is placed in PMMA. Previous studies using PMMA as the host polymer for EO chromophores have shown that poling needs to take place at temperatures of at least 85° C. to have chromophore mobility, where we have shown chromophore mobility at least 60° C. below the standard accepted temperature.

TABLE 15
Summary of the EO response of a small EO chromophore

| Temperature | Bias field | EO |
|---|---|---|
| 25° C. | 25 V/μm | 1.47 pm/V |
| 29° C. | 25 V/μm | 1.74 pm/V |
| 33° C. | 25 V/μm | 1.94 pm/V |
| 37° C. | 25 V/μm | 2.17 pm/V |
| 41° C. | 25 V/μm | 2.39 pm/V |
| 45° C. | 25 V/μm | 2.6 pm/V |
| 45° C. | 35 V/μm | 3.64 pm/V |
| 45° C. | 45 V/μm | 4.7 pm/V |
| 45° C. | 55 V/μm | 5.83 pm/V |
| 45° C. | 65 V/μm | 7.02 pm/V |
| 45° C. | 75 V/μm | 8.1 pm/V |

The EO response of this material to EO Profile2 is shown in FIG. 8. For clarity, the temperature profile is not shown in this Figure. The material is heated from time 0 to approximately time 6 minutes, then allowed to cool to room temperature. The material reaches room temperature before the bias field is removed around time 13 minutes. The bias field of 50 V/μm corresponds to a 1000V bias across the 20 μm gap between the electrodes. The EO response has a value of approximately 30 pm/V when heated to 55° C., with the response dropping to about 15 pm/V at room temperature

The stability of the response to constant illumination at 405 nm has been tested by measuring the EO response of a second test cell, then removing the applied voltage and continuing to illuminate the sample with the 405 nm source over the course of the weekend. Measurement of the response after more than 63 hours of illumination showed no change in the material response outside the range of experimental error (from 3.4 pm/V to 3.5 pm/V).

### Example 8

### EO Properties of Small EO Material (178-096-21)

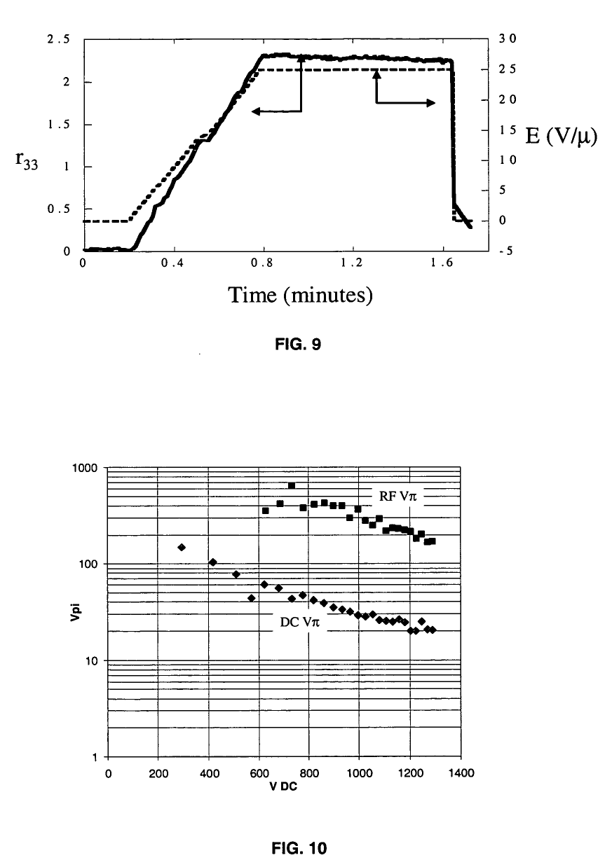

In this example we present the EO response of small chromophore 4DMABN. This chromophore was placed at 20.14 wt-% in Lumiflon LF-910-LM (Asahi Glass Company). The response of the chromophore at 1 kHz using EO Profile2 is shown in FIG. 9, using a 1550 nm laser as the optical source. The sample was held at 20° C. for the entire test. Here we see there is a strong response at room temperature. The EO response falls off very rapidly when the bias voltage is removed, with only a small fraction remaining at zero volts. This residual EO then quickly dissipates. Again, this effect is happening far below the glass transition temperature of the polymer.

### Example 9

### MZI Device Fabricated Using Standard Chromophore/Polymer Blend (Yankees1)

In this example we present the results for a MZI device fabricated using a material composed of 8.55 wt-% VC8 in CP087. The EO material in a dioxane solution (˜12% solids) was coated onto a silica MZI substrate, then dried and fabricated into an optical device (Yankees1) using standard OPI fabrication procedures. An EX electrode was placed on the device, giving a total electrode interaction length of 4 cm. The device was heated to so the refractive index of the EO material was less than that of the silica waveguide core so guiding would occur, and 1550 nm TE light was input into the Mach-Zehnder. The DC voltage of the electrodes was then ramped, and the output power was monitored as a function of voltage. The difference in the voltage between each of the quadrature points, where the optical power is midway between its maximum and minimum values, provides the value of the DC Vπ. This value measures the total birefringence, orientational and electronic, induced in the device by the application of the slowly ramped electric field. At any of the quadrature points, an oscillatory signal can also placed on the electrodes, using a range of frequencies. From measuring the magnitude of the response at that frequency and knowing the voltage of the applied oscillatory signal, we compute the voltage, or RF Vπ, needed to completely drive the MZI from fully on to fully off at that frequency, with that DC bias voltage.

The two Vπ values can be used to determine the ratio between the electronic component of the birefringence and the electronic plus orientational birefringence. At very high frequencies (>100 MHz) the response is purely electronic in nature, and the magnitude of the response is inversely related to the RF Vπ. (The results in Example 2 demonstrated that conventional chromophores have difficulty orientating in response to a 1 kHZ signal, so 1 100 MHz signal is orders of magnitude faster than chromophore reorientation can occur for these conventional chromophores.) At very low frequencies (DC) the material responds to the voltage with the orientational plus the electronic components, and the magnitude of this response is inversely proportional to the DC Vπ. Thus the ratio at high frequency of the RF Vπ to the DC Vπ directly measures the ratio of the electronic to electronic plus orientational response of that chromophore. The prior art has established this high frequency as 1 kHz. By varying the frequency at which this ratio is measured, it is also possible to study the transition of the material from the purely electronic response of an oriented material to that of a material orienting in response to a slowly changing voltage.

The response of the device at RF frequencies was determined by applying both a DC and RF voltage to the electrodes. The DC voltage is then ramped, and at each quadrature point, the RF Vπ is determined from the change in optical power at the RF frequency. For this example, the RF frequency was chosen to be 480 MHz. The response at DC and RF for different bias voltages is shown in FIG. 10, for this device operating at 78° C., well above the temperature needed for chromophore orientation in this material.

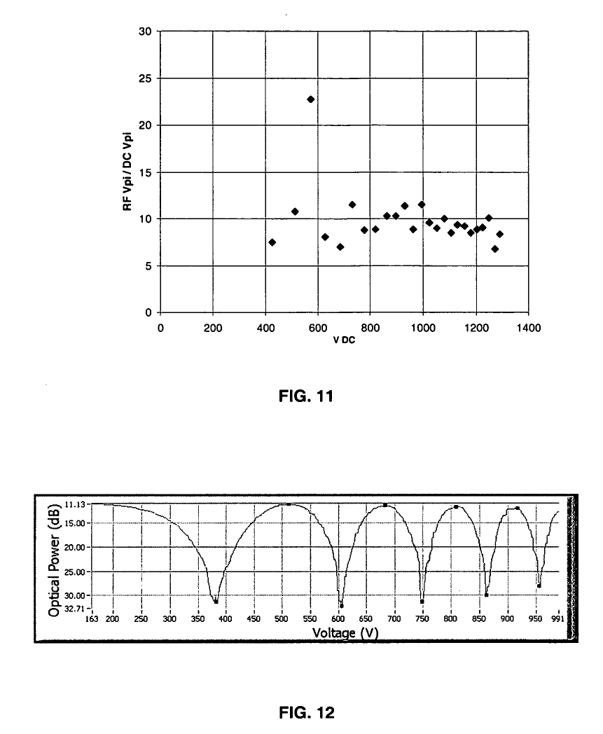

The ratio of these two responses, shown in FIG. 11, determines the relation between the purely electronic and the electronic plus orientational components of the response. Although these is some scatter in the plot, this ratio is seen to be approximately 10, about what is expected for a typical conventional EO chromophore. As remarked before, conventional chromophores with lower FOM values have been measured to have ratios around 17, and this ratio is expected to decrease with increasing quality of the conventional EO chromophore. Note also that this ratio is relatively constant for all DC bias voltages, as is expected from the theory of the response of these devices to applied voltage.

### Example 10

### Silica MZI Device Clad with 4DMABN/CP087 (Allspice1)

A solution of 21.61% 4DMABN in CP087 (178-090-21) was coated onto a silica MZI substrate, then dried and fabricated into an optical device (Allspice1) using standard OPI fabrication procedures. An EZ electrode was placed on the device, giving a total electrode interaction length of 3 cm. The device was heated to 45° C., and 1550 nm TE light was input into the Mach-Zehnder. The waveguides and splitters on the device were designed for use with 1550 nm light, and did not function ideally with 405 nm light. The voltage of the centered electrodes was then ramped, and the output power was monitored as a function of voltage. FIG. 12 shows the response of the device to 1550 nm light. The results show the device has a high extinction ratio, and good optical throughput.

### Example 11

### Sol-Gel MZI Device Clad with 4DMABN/CP087 (178-084-33#3)

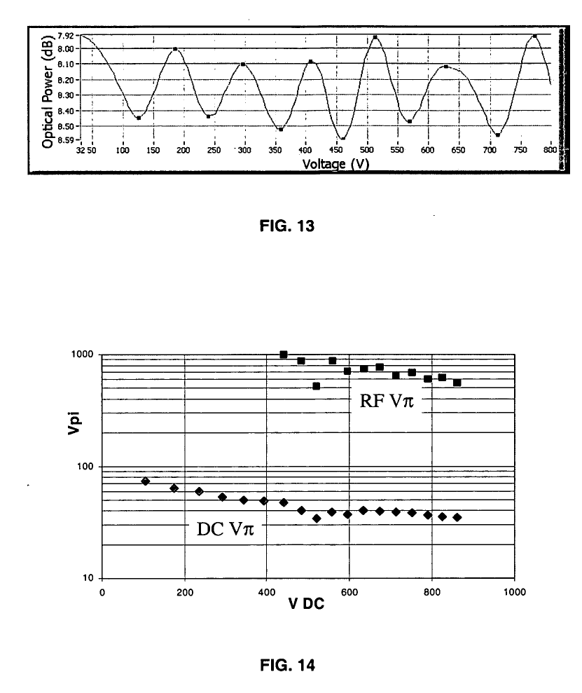

Optical quality sol-gel waveguides were obtained (Japan Synthetic Rubber) or made according to the recipes and processes described in “Sol-Gel Technologies for Thin Films, Fibers, Preforms, Electronics, and Specialty Shapes”, Lisa C. Klein ed., Noyes Publications, Park Ridge, N.J., 1988. A solution of 21.56% 4DMABN in CPO87 (178-084-33) was coated onto a sol-gel MZI substrate, then dried and fabricated into an optical device (178-084-33#3) using standard OPI fabrication procedures. An EW electrode was placed on the device, with an electrode interaction length of 3 cm. The device was heated to 60° C., and 1550 nm TE light was input into the Mach-Zehnder. The voltage of the electrodes along one arm was then ramped, and the output power was monitored as a function of voltage. FIG. 13 shows the response of the device to 1550 nm light. The results show the device has a high extinction ratio, and good optical throughput. Previous work with conventional chromophores and sol-gel substrates have shown the chromophore penetrates the sol-gel, thus placing chromophore directly into the waveguide. This has the effect of enhancing device performance in the sol-gel devices using conventional chromophores. The penetration of the high-speed chromophore into the sol-gel matrix may also enhance the device response for these devices based on the high-speed materials of this invention.

The response of the device at RF frequencies was then determined by applying both a DC and RF voltage to the electrodes. The DC voltage is then ramped, and at each quadrature point (where the power is midway between its maximum and minimum), the RF Vπ is determined from the change in optical power at the RF frequency. For these tests, the RF frequency was chosen to be 480 MHz. The response at DC and RF for different bias voltages is shown in FIG. 14.

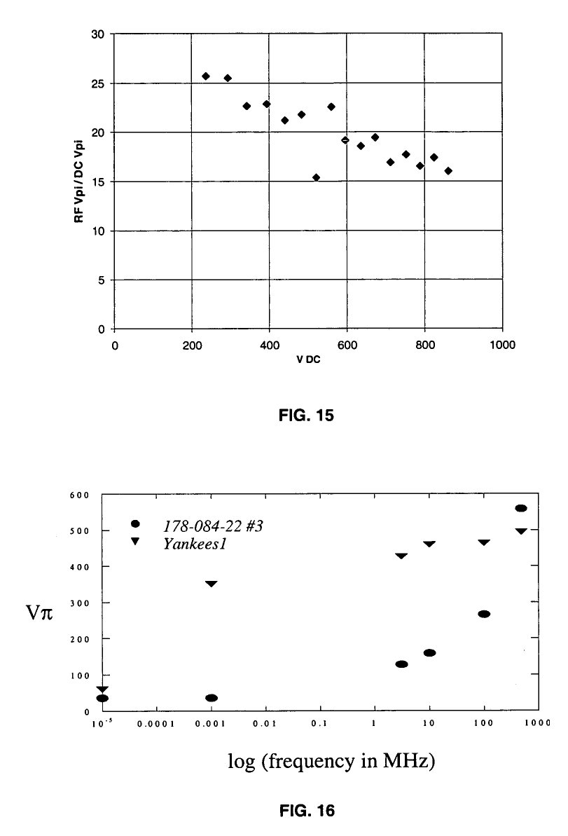

The ratio of these two responses, shown in FIG. 15, determines the relation between the purely electronic and the electronic plus orientational components of the response. There are two substantial differences between FIG. 15 for the 4DMABN chromophore and FIG. 11 for the VC8 chromophore. The first is that the ratio of the two responses is much larger, as would be expected for a chromophore with a much smaller electronic response (smaller PFOM), as is the case with 4DMABN. The second difference is that this ratio is not constant, but decreases with increased DC bias voltage.

The response of the device at 405 nm can be approximated using the dispersion relation described previously. Using the response at 1550 nm and the absorption maximum of 4DMABN of 290 nm, the EO response at 405 nm should be approximately 3.3 times that measured at 1550 nm, or the RF Vπ at 405 nm should be about 0.3 times the RF Vπ at 1550 nm.

### Example 12

### Frequency Dependent Response of Yankees1 and 178-084-33#3 Devices

The response of standard EO chromophores and those of this invention differ in their response to higher frequency voltage. To demonstrate this, both the Yankees1 and 178-084-33#3 devices were tested with voltage across a range of frequencies. The Vπ response at a DC bias voltage of approximately 900V was recorded, and is shown in FIG. 16. The frequencies in MHz are plotted on a logarithmic axis to readily accommodate the wide range of frequencies. The DC Vπ is arbitrarily assigned a frequency of 1 Hz to allow its inclusion, since the response of all these materials at 1 Hz should be identical to its purely DC response. The response of the Yankees1 device at 1 kHz is estimated from the EO cell response shown in Example 2.

The data shown in FIG. 16 can be readily summarized as the conventional EO material (Yankees1) shows a large response (low Vπ) at very low frequencies, but by 1 kHZ the response is almost purely electronic in nature, with the response remaining nearly constant across the range of 1 MHz and above. In contrast, the material of this invention (178-084-33) shows large response (low Vπ) across an extended frequency range, with the response at 3 MHz still much lower than that of the Yankees1 device. Only at 480 MHz does the performance of the Yankees1 device exceed that of the 178-084-33#3 device.

It is expected at even higher frequencies, the Yankees1 performance would continue at the same level while that of the 178-084-33#3 device would continue to deteriorate until its Vπ is several times that of the Yankees1 device. Based on the chromophore FOM values and the relative concentrations, we would expect the ultra-high frequency performance of Yankees1 to be approximately 8 times better than that of the 178-084-33#3 device. Limitations of the device substrate prevent the testing of 178-084-33#3 at frequencies higher than 480 MHz.

### Example 13

### Response of 178-084-33#3 to a Rapid Voltage Step

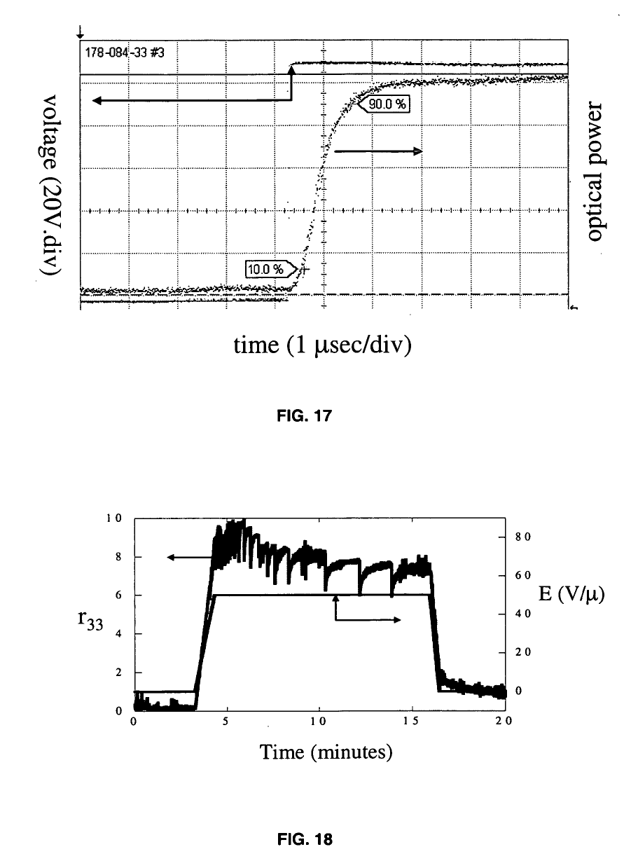

A separate demonstration of the rapid response of the materials of this invention is given in this example. In this case, an ultrafast (<1 μsec) voltage step is applied to the device, and the response measured as a function of time. FIG. 17 shows an oscilloscope trace of the voltage step and the optical power output of the device. Here we see the rapid voltage step of about 120V and the switching of the optical power within 1 μsec. Again, because of the small optical nonlinearity of this chromophore, this switching is performed by rotation of the chromophore molecules within 1 μsec in response to the change in the applied voltage. This is verified by the voltage step required to perform this switching. At this temperature, the 125V step applied is less than twice the DC voltage required to create this change in the optical power, while a purely electronic response would require a voltage more than 10 times larger than the required DC voltage.

### Example 14

### Use of Other Substrates

The high-speed materials of this invention can also be incorporated onto alternate optical substrates. The examples given previously utilized these materials as the functional cladding on MZI devices fabricated from silica or sol-gel. The materials of this invention can be utilized equally well as the functional cladding of optical devices made with other materials, such as sol-gel glasses, SiON, or polymers as examples. The primary constraints are those stated previously, that the refractive index of the clad must be lower than that of the substrate at the operational temperature, and that the materials must be compatible with the substrate. The high speed materials of this invention are also suitable for use as the active core or active core and cladding for optical devices.

### Example 15

### Use of Other Host Materials

The high-speed materials of this invention can also use hosts other than the thermoplastic polymers of the previous examples. For example, chromophores of the type described above may be incorporated in the following:

Solvent Swollen or Plasticized Thermoplastic Polymers

In this case a thermoplastic polymer or copolymer is added to a compatible solvent (1%-99% polymer or 99%-1% solvent) that contains a chromophore of the type described above.

Crosslinked

Polymers containing reactive functionalities that can be crosslinked in the presence of a reactive crosslinking agent or reactive or non-reactive solvents with chromophores of the type described above. The crosslinking may occur via thermal, UV, or other methods.

Direct Formation

The material is formed and crosslinked in situ using combinations of reactive monomers, polymers, and solvents in the presence of chromophores of the type described above. The crosslinking may occur via thermal, UV, or other methods.

Interpenetrating Polymer Networks

Any combination of the three previous methods.

Sol-Gel Matrices

The material is formed by directly incorporating chromophores of the type described above directly into the sol-gel prior to the baking or UV exposure that is the final curing step. The sol-gel may be fully inorganic, or may incorporate some organic functionality. Alternately, the chromophore may be introduced into the sol-gel after final curing by vapor deposition, solution deposition, diffusion, or other means to diffuse chromophore molecules of the type described above into the sol-gel matrix.

In all these examples, the interaction between the host matrix and the chromophores must be sufficiently weak to allow for chromophore rotation at with characteristic times of under 1 microsecond. For crosslinked materials, this may set a functional limit on the allowable crosslink density.

### Example 16

### Loss Measurements of EO Materials at 405 nm

In this example we present the loss measurements for 405 nm light propagating through films of the following three materials, 4DMABN/PMMA, 4DMABN/CP087, and 4A2TFMBWCP087, all at approximately 21 wt-% chromophore. For all three solutions, a film approximately 6 microns in thickness was cast upon a quartz slide. This film thickness is sufficient for the film to act as a bulk waveguide. After drying, 405 nm light was prism-coupled into and out of the film. By varying the separation between the two prisms, an approximate value for the propagation loss in the material is obtained. The results of the measurements are shown in Table 16.

TABLE 16
Loss Measurements for Assorted Materials at 405 nm

| Material | Loss |
|---|---|
| 4DMABN/PMMA | 9 dB/cm |
| 4DMABN/CP087 | 11 dB/cm |
| 4A2TFMBN/CP087 | 1 dB/cm |

### Example 17

### EO Properties of Small EO Material (178-084-02)

In this example we present the results of EO measurements made on a solution of 21.5% 4DMABN in CP087. The solution was coated onto an EO cell, and measurements made using the procedure described above. The solution was also coated onto a prism for a refractive index measurement. The value of the index was measured to be 1.4549 at room temperature and 1550 nm.

The EO response of the material at 405 nm is shown in FIG. 18. The material is heated from time 0 to approximately time of 6 minutes, then allowed to cool to room temperature. The material reached room temperature before the bias field was removed. The EO response has a value of approximately 9 pm/V when heated to 45° C., with the response dropping to about 7 pm/V at room temperature. The response at higher temperatures is larger, but there is a larger chance of the EO cell shorting at these temperatures.

### Example 18

### Melting Points of Blends (Eutectics) of Chromophores (141-127-02)

In this invention we have discovered that combinations of two or more chromophores can lower the melting point of the highest melting chromophore, and this combination allows for greater solubility in the polymer (better film and optical properties) and improved NLO response than the individual components alone.

Several combinations of a high melting chromophore (4-aminophthalonitrile, melt point 179°-181° C.) and higher or lower melting chromophores were formed by the following procedure. In a 2-dram vial were placed the noted amounts of 4APTN (m.p. 179°-181° C.) and the second compound (X.) The solids were melted using a hot plate and allowed to cool. A small amount of the resulting material was transferred to the melting point apparatus stage and a melting point was taken. The results are given in Table 17.

TABLE 17
Melting Points of Chromophore Blends

| | | | Weight of | Mixed Melting | |
|---|---|---|---|---|---|
| | Melting Point of X | Weight of X | 4APTN | Point A/X | |
| Compound X | (degrees C.) | (mg) | (mg) | (degrees C.) | Results |
| 4A2TFMBN | 136-139 | 10.3 | 10.6 | 121-131 | Waxy solid, creamy brown color, darker solids |
| | | | | | melted at 120-121, lighter solids melted from |
| | | | | | 121 to 131. |
| 3F6MPBN | 93-95 | 10.8 | 10.3 | 79-80 | Slightly yellow solid |
| 2F6MPBN | 92-94 | 10.5 | 10.8 | 116-144 | Off-white solid began softening at 116, turned |
| | | | | | into a slush at 131, and was finally liquid at |
| | | | | | 144. |
| DMAMMN | 84-85 | 9.9 | 9.6 | 81-86 | Off-white solid |
| 4DMABN | 73-75 | 10.2 | 10.6 | 56-58 | Yellow solid |
| 3A5NBT | 250 | 10.4 | 10.6 | 168, 178-202 | Red solid, cloudiness* appeared < 150, a very |
| | | | | | small number of particles melted at 168, the |
| | | | | | bulk of the material melted from 178-202. |
| 2A6NBT | 247-249 | 10.6 | 10.8 | 156-164 | Brown solid, cloudiness* appeared < 150 C., |
| | | | | | solids melted from 156-164. |
| 2A5NP | 235-239 | 10.4 | 10.5 | 157-158 | Orange solid |
| 2A4M5NP | 223 | 10.2 | 10.3 | 152-155 | Waxy brown solid |
| 2A5NPST | 222-226 | 10.4 | 10.4 | 152-154 | Yellow solid |

*The cloudiness mentioned in the table is a white condensation that completely surrounds all the solid particles. It is almost as though something is subliming and condensing out on the top cover slip. In all cases, the cloudiness disappears as heating is continued.

### Example 19

### Chromophore Preparation and Evaluation

In this example, a new chromophore was synthesized and evaluated. This chromophore was also converted into a monomer and reacted with methyl methacrylate to form an electrooptic polymer, which was also evaluated. The chromophore and polymer were prepared according to the following schemes. The references associated with reaction schemes A, B, and C are:
A) Reactive and Functional Polymers, vol. 30, pp 375-383 (1996).
B) Adapted from Organic Synthesis Collective, vol IV, N. Rabjohn ed., p 478, J. Wiley, New York, 1963.
C) Preparative Methods of Polymer Chemistry, W. R. Sorenson and T. W. Campbell, pp 183-184, Interscience Publisher, New York, 1961.

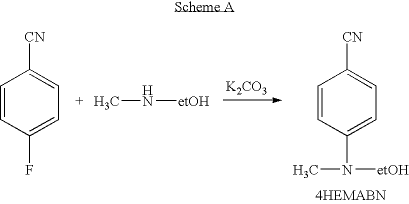

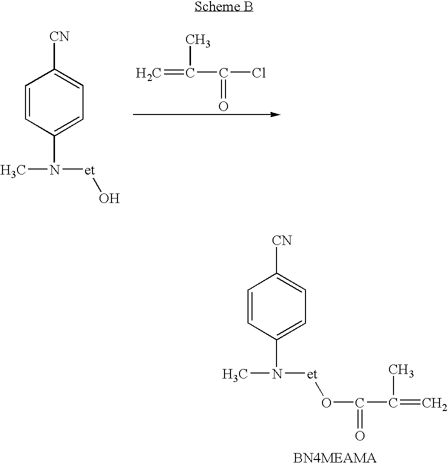

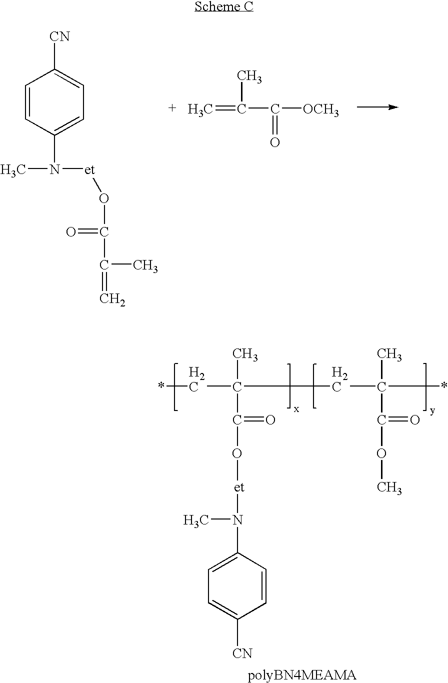

The EO response for 4HEMABN at 20 wt-% in CP087 was 7 pm/V. The EO response for a 80/20 copolymer of MMA and BN4MEAMA (x=0.2, y=0.8) was 10 pm/V. Broadly, x can range from about 10 to 100 mole fraction (mol-%), while y can range from about 0 to about 90-mole fraction (mol-%). Thus homopolymers and copolymers of acrylate esters of the polymerizable monomer, BN4MEAMA, are new compounds disclosed herein.

### Example 20

### Optical Quality and EO Response of Blends (Eutectics) of Chromophores

In this example, several different blends of chromophores were evaluated for their film forming properties, using the previously described methods. Additional blends were evaluated for the EO response of the system. The data shows that using a blend of two chromophores in an EO material can lead to higher optical quality films than would be obtained by using an equivalent amount of a single chromophore. The EO results at both 405 nm and 1550 nm show that blending two chromophores can greatly enhance the EO response of the material over that expected for either of the two components alone in the polymer at the same total concentration.

TABLE 18
Film Quality of Blends and Their Components

| | Film |
|---|---|
| Composition | quality rating |
| 20 wt-% 4DMABN in CP087 | 3 |
| 20 wt-% 4APTN in CP087 | 5 |
| 20 wt-% 5A2CBTF | 5 |
| 10 wt-% 4DMABN and 10 wt-% 4APTN in CP087 | 3 |
| 10 wt-% 4DMABN and 10 wt-% 5A2CBTF in CP087 | 3 |

TABLE 19
EO Response of Blends at 405 nm

| | | Chromo- | | |
|---|---|---|---|---|
| Solution | Chromophore 1 | phore 2 | Polymer | EO result |
| 203-009-12 | 10% 4DMABN | 9.58% | CP093 | 6.7 pm/V |
| | | 4APTN | | |
| 203-013-16 | 11.88% 4DMABN | 8.13% | CP093 | 11.65 pm/V |
| | | 4APTN | | |
| 203-013-26 | 10.02% 4DMABN | 10.5% | PCARB | 18.0 pm/V |
| | | 4APTN | | |
| 203-009-16 | 9.47% 4DMABN | 9.88% | CP093 | 10.0 pm/V |
| | | 4A2TFMBN | | |
| 203-013-20 | 10.07% 4DMABN | 10.07% | CP093 | 9.8 pm/V |
| | | 4A2TFMBN | | |

TABLE 20
Refractive Index and EO Response of Blends at 1550 nm

| | | | | | Refractive | | |
|---|---|---|---|---|---|---|---|
| Solution | Chromophore 1 | | Chromophore 2 | Polymer | Index | EO result | |
| 178-104-14 | 10.2% | 4DMABN | 10.7% 4N3TFMA | CP087 | 1.459 | 22 | pm/V |
| 203-033-02 | 11.37% | 4DMABN | 10.7% 4N3TFMA | CP073 | 1.4557 | 13.8 | pm/V |
| 178-118-06 | 10.41% | MNA | 10.02% 4N3TFMA | CP087 | 1.462 | 4.0 | pm/V |
| 178-093-24 | 21.5% | 4DMABN | — | CP087 | | 10.0 | pm/V |
| 178-093-06 | 20.0% | 4N3TFMA | — | CP087 | | 8.1 | pm/V |
| 178-124-10 | 9.97% | MNA | — | CP087 | | 11.0 | pm/V |
| 178-130-20 | 14.6% | 4DMABN | — | CP073 | | 8.3 | pm/V |
| 178-130-32 | 15% | 4N3TFMA | — | CP073 | | 7.6 | pm/V |

### Example 21

### Optical Quality and Guiding in Bulk Waveguides

Bulk waveguides were fabricated and tested as described previously using several different solutions. All films were approximately 3 microns in thickness. Either 405 nm or 633 nm laser light was prism coupled into the EO material, and after traversing approximately a 1 inch path, the light was then prism coupled out of the waveguide. The intensity and shape of the output spot was then evaluated to determine the optical quality of the film.

TABLE 21
Guiding Quality at 633 nm

| Solution | Chromophore | Polymer | Results |
|---|---|---|---|
| 203-091-24 | 15.08% 4DMABN | CP061 | good guiding, nice spot |
| 203-094-05 | 21.71% 4HEMABN | CP061 | good guiding, nice spot |
| 203-091-07 | 21.50% 4N3TFMA | CP087 | no guiding |
| 203-091-21 | 19.98% 4N3TFMA | CP061 | strong guiding, nice |
| | | | spot |
| 203-091-27 | 19.96% 4N3TFMA | CP050 | good guiding, nice spot |
| 203-094-25 | 18.06% 4HEMABN | SPIKE | no guiding, cracks |
| | | | under the prsms |
| | | PMMA | hazy, grainy, streaky |
| | | | spot |

TABLE 22
Guiding Quality at 405 nm

| Solution | Chromophore | Polymer | Result |
|---|---|---|---|
| 203-068-15 | 21.46 4DMABN | CP087 | good guiding, streaky spot |
| 203-068-18 | 21.52 4DMABN | SPIKE | good guiding, streaky spot |
| 203-074-12 | 21.46 4DMABN | PMMA | guides, poor spot |
| 203-091-24 | 15.08 4DMABN | CP061 | guides a short distance, no |
| | | | output spot |
| 203-094-05 | 21.71 4HEMABN | CP061 | guides a short distance, no |
| | | | output spot |
| 203-094-25 | 18.06% 4HEMABN | SPIKE | no guiding, cracks under |
| | | | the prsms |
| 203-091-15 | | CP061 | nice clear spot |

These results show that obtaining optical quality films for 405 nm light transmission involves more than selecting a chromophore, which does not absorb at 405 nm. Because scattering is enhanced at short wavelengths, good compatibility between the chromophore and polymer is vital for guiding at short wavelengths. The data in the table shows the guiding for 4DMABN in four different polymers varies greatly, even though the polymers themselves support good guiding at 405 nm. The situation is the same at 633 nm, where the compatibility between the chromophore and polymer strongly impacts the guiding quality of the film.

### Example 22

### Additional Chromophore/Polymer Blends

In addition to the previously described materials, the following have also chromophore/polymer blends have been studied for their film formation or electrooptic properties.

TABLE 23
EO Response of Chromophores at 405 nm

| Sample | Concentration | Chromophore | Polymer | EO (pm/V) |
|---|---|---|---|---|
| 178-090-25 | 21.59 | 4A2TFMBN | CP087 | 18.2 |
| 178-084-25 | 20.24 | 4APTN | PMMA | 8.5 |
| 178-072-16 | 20.16 | 4DMABN | CP087 | 13 |
| 203-022-19 | 51 | 4DMABN | PCARB | 24.2 |
| 178-084-10 | 21.7 | 4DMABN | PMMA | 18.1 |
| 203-022-15 | 58.6 | 4DMABN | PMMA | 22.3 |
| 178-084-06 | 18.0 | NB | PMMA | 30 |

TABLE 24
EO Response of Chromophores at 422 nm

| Sample | Concentration | Chromophore | Polymer | EO (pm/V) |
|---|---|---|---|---|
| 178-072-22 | 11.75 | 4N3TFMA | SPIKE | 2.3 |
| 153-134-14 | 10.55 | MNA | CP076 | 3.1 |

TABLE 25
EO Response of Chromophores at 633 nm and 830 nm

| Sample | Concentration | Chromophore | Polymer | EO (pm/V) |
|---|---|---|---|---|
| 178-084-02 | 21.5 | 4DMABN | CP087 | 6.7 |
| 203-091-30 | 20.91 | 4N3TFMA | CP050 | 7.2 |
| 203-094-34 | 20.06 | 4N3TFMA | CP061 | 17.2 |
| 178-096-29 | 20 | 4N3TFMA | CP091 | 8.2 |
| 203-003-05 | 21.63 | 4DMABN | CP087 | 8.2 |
| | | | | (830 nm) |

TABLE 26
EO Response of Chromophores 1550 nm

| Sample | Concentration | Chromophore | Polymer | EO (pm/V) |
|---|---|---|---|---|
| 178-062-06 | 12.32 | 2,5-BTFMA | CP087 | 0.12 |
| 178-100-31 | 19.18 | 35DMOBN | CP087 | 2.2 |
| 178-080-26 | 19.7 | 4A2TFMBN | CP087 | 5.8 |
| 178-115-10 | 21.06 | 4A3BN | CP087 | 6 |
| 178-062-10 | 10.85 | 4A4NPS | CP089 | 3.2 |
| 178-135-19 | 14.89 | 4ABN | CP073 | 7.2 |
| 178-143-10 | 19.69 | 4APTN | PMMA | 1.2 |
| 178-118-10 | 21.52 | 4DMABN | CP029 | 12 |
| 178-096-33 | 19.73 | 4DMABN | CP051 | 11.7 |
| 178-130-20 | 14.64 | 4DMABN | CP073 | 8.3 |
| 178-090-21 | 21.61 | 4DMABN | CP087 | 10.9 |
| 178-093-24 | 21.53 | 4DMABN | CP087 | 10 |
| 178-104-02 | 20.15 | 4DMABN | CP093 | 6.4 |
| 203-010-25 | 21.02 | 4DMABN | PMMA | 18 |
| 203-091-34 | 20.21 | 4HEMABN | CP061 | 16.4 |
| 203-083-12 | 21.08 | 4N3TFMA | CAB | 6.2 |
| 203-091-30 | 20.91 | 4N3TFMA | CP050 | 8.2 |
| 178-093-06 | 20 | 4N3TFMA | CP087 | 8.2 |
| 178-096-29 | 20 | 4N3TFMA | CP091 | 10.4 |
| 178-093-09 | 20.5 | 4N3TFMA | CP093 | 7.4 |
| 178-096-09 | 19.75 | 4N3TFMA | SPIKE | 5.2 |
| 178-108-11 | 9.62 | 4NA | CP087C | 4 |
| 178-062-30 | 10.14 | 4NA | PMMA | 2.9 |
| 178-062-02 | 12.29 | 4-TFMA | CP087 | 0.5 |
| 178-115-26 | 20.16 | DNPA | CP087 | 7.4 |
| 178-135-23 | 14.72 | MNA | CP073 | 4.2 |
| 153-134-14 | 10.55 | MNA | CP076 | 10.5 |
| 178-124-10 | 9.97 | MNA | CP087 | 11 |
| 178-138-26 | 10.01 | MNA | CP087 | 10 |
| 178-135-02 | 20.66 | MSA | CP087 | 0.2 |

### Example 23

### EO Response at 633 nm and 830 nm

The EO response of two chromophores was evaluated at both 633 nm and 830 nm.

TABLE 27
EO Response at varying wavelengths

| Composition | Wavelength | EO response |
|---|---|---|
| 21.6 wt-% 4DMABN in CP087 | 633 nm | 4.9 pm/V at 37 C. |
| 21.6 wt-% 4DMABN in CP087 | 830 nm | 9.0 pm/V at 50 C. |
| 11.98 wt-% EC24 in CP087 | 633 nm | 16.1 pm/V at 60 C. |
| 11.98 wt-% EC24 in CP087 | 830 nm | 12.9 pm/V at 55 C. |

The response of EC24 at 633 nm was enhanced by resonance effects (the absorption maximum for EC24 is 503 nm).

### Example 24

### Response of Silica MZI Device (Odyssey3) Clad with a Conventional EO Material (EC23/CP087) to a Rapid Voltage Step

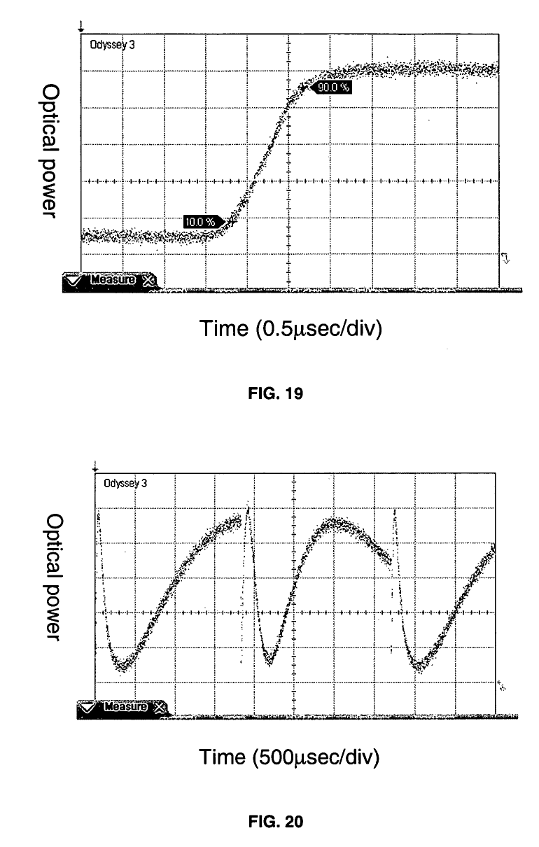

A demonstration of the rapid response of a conventional EO polymer material is given in this example. The MZI device was fabricated using a material composed of 11.9 wt % EC23 (4-dimethylaminophenyl-1,3 carboxyethyl,4,4 dicyanobutadiene) in SPIKE. The EO material in a dioxane solution (˜12% solids) was coated onto a silica MZI substrate, then dried and fabricated into an optical device (Odyssey3) using standard OPI fabrication procedures. An EX electrode was placed on the device, giving a total electrode interaction length of 4 cm. An ultrafast (<1 μsec) voltage step was applied to the device, and the response was measured as a function of time (FIG. 19). Here we see the rapid voltage step of about 250V and the switching of the optical power within 1 μsec. Here, the large voltage required to switch this device within 1 μsec demonstrates that the response is primarily electronic in nature, not orientational. The DC voltage required for this change in optical power is less than 50V, while the RF voltage required is approximately 500V. A second demonstration of the primarily electronic response of this material at short times is shown in FIG. 20, where the response to the step voltage is shown for times up to 2 msec. In this case, the optical power continues to vary as the voltage is held constant, consistent with the chromophore molecules slowly reorienting in response to the changed voltage. Notice that the power indicates a much large change in phase over the course of the 2 msec time than was seen in the initial rapid response. This is in contrast to the materials of this invention, where after the initial response within 1 msec, the power remains relatively constant over the next several microseconds, indicating that the chromophore reorientation almost completely occurs within the initial time period.

### Example 25

### Blends of Conventional Chromophores with the Small Chromophores of This Invention

In this example, we demonstrate that mixing a small amount of a small chromophore, as described in this invention, into a EO material consisting of a polymer and conventional EO chromophore. Two samples were prepared and used to fabricate EO cells. The first sample was 7.99 wt % VC8 in CP093 (178-110-06), the second was 7.72 wt % VC8 and 0.50 wt % 4DMABN in CP093 (178-126-18). After drying, EO measurements were performed on each sample, using Profile 2. The conventional sample needed to be poled at 70 C, and returned an RFEO of 22.6 pm/V. The sample which included the small chromophore needed to be poled at 50 C and returned an RFEO of 27.4 pm/V. The “Poled EO” of the sample including the small chromophore diminished rapidly at room temperature, while that of the conventional material remained large over the same time period. Thus inclusion of the small chromophore greatly improved the mobility of the conventional chromophore and enhanced its ability to align in the presence of an applied field, increasing its EO response. This synergistic response between two chromophores has also been observed in blends of conventional chromophores, where combining two or more chromophores can lead to improved film quality and larger EO response compared to a system where there is an equal amount of only one of the chromophores.

### Example 26

### Fast Photorefractive Polymer Composite

The photorefractive effect consists of a spatial modulation of the refractive index of a photorefractive composite induced by an inhomogeneous light pattern. In low Tg polymer photorefractive materials, orientation of the chromophores is the primary mechanism for generating the large, spatially varying, birefringence of the photorefractive composite. One of the best photorefractive composites consists of a photoconductive poly(N-vinylcarbazole)/N-ethylcarbazole matrix sensitized with (2,4,7-trinitro-9fluorenylidene)malonitrile, then doped with one of many possible chromophores (K. G. Jesperson, et. al., J. Opt. Soc. Am. B 20, 2179-2188 (2003); D. Van Steenwinckel, et. Al., J. Chem. Phys. 112, 11030-11037, (2000); K. Hoechstetter, et. al., J. Chem. Phys. 110, 4944-4951 (1999); J. A. Herlocker, et. al., Appl. Phys. Lett. 74, 2253-2255 (1999); L. Mager, et. al., Appl. Phys. Lett. 71, 2248-2250 (1997); B. Swedek, et. al., J. Appl. Phys. 82, 5923-5925, (1997); B. Kippelen, et. al., Appl. Phys. Lett. 68, 1748-1750 (1996)).

One of the limiting factors in the performance of photorefractive polymers is the speed of the response, which is typically measured on the millisecond time frame. In fact, the typical method used to determine the separate the contribution of the electronic and orientational components of the photorefractive response is the measure the response at a low frequency, where both components contribute, and at a high frequency, where there is no orientational component. In the literature, this high-speed measurement takes place at a frequency of 1 kHz. This indicates that the response for these photorefractive composite materials must slower than 1 ms.

A photorefractive composite material which uses one of the chromophores of this invention in place of a standard photorefractive chromophore, as described in the prior art, will have a response time consistent with the demonstrated response time of less than 1 microsecond. Thus photorefractive materials using the chromophores of this invention will have performance far exceeding that of the prior art.

### Example 27

### Bragg Grating Modulator

A second type of EO modulator was fabricated in the following manner. An approximately 3 micron thick lower cladding consisting of a thermally-crosslinked low index polymer was spun onto a fused silica substrate. Onto this an approximately 3 micron thick layer consisting of 4HEMABN in CP087 was spun and dried. A Bragg grating with 8 micron gap between the electrode fingers was formed on a separate fused silica cover plate. An approximately 2 micron thick layer of SPIKE polymer was spray-coated onto the electrode plate to form an upper cladding. The electrode cover plate was then placed on top of the EO polymer to form a waveguide structure. 633 nm light was prism coupled in and out of the device. Application of voltage to the electrodes of the Bragg grating caused the appearance of diffracted light at the Bragg angle for the grating. 405 nm light could also be coupled into and out of this waveguide, with a corresponding decrease in the Bragg diffraction angle.

### Example 28

### Application of 405 nm Modulation for DVD Application

405 nm radiation is of interest for many applications, including data storage, where the smaller wavelength of radiation allows for an increase in data density. For many applications, this increase in data density is accompanied by a need for increased speed in writing the data. In particular, the data writing rate required for real-time recording of high-definition television exceeds that possible by the methods used for writing conventional DVDs. One application of the 405 nm EO materials disclosed in this invention is to create devices to modulate 405 nm light at high speeds, allowing for the rapid writing of data in high-density, high data rate applications.

### Example 29

### Application of Visible and Near-IR Modulation

Laser printers work by modulating an input laser beam, while simultaneously moving the focused laser beam. Increases in the resolution of the printer (while maintaining the same or a faster print rate) require an increase in the modulation rate of the laser while decreasing the size of the spot. Because they are capable of rapidly modulating visible and near-IR radiation, the materials of this invention can enable new or enhanced devices using short wavelength radiation.

While the invention has been described with reference to specific embodiments, those skilled in the art will understand that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed, but that the invention will include all embodiments falling within the scope of the appended claims.

We claim:

1. An electrooptic chromophore for use in forming an electrooptic material (EO) comprising an electrooptic polymer and an electrooptic chromophore, which EO operates at about 405 nm, which comprises: 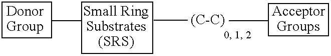
wherein, said electrooptic chromophore has a weak absorption at about 405 nm and a maximum absorption at less than about 330 nm.

2. The electrooptic chromophore of claim 1, wherein:

| Donor | SRS | Acceptor |
|---|---|---|
| Weak | Light or Medium | Weak, Medium, or |
| | | combination |
| Strong | Light or Medium | One or more weak |
| Weak | Heavy | One or more weak |

3. The electrooptic chromophore of claim 2, said donor group, said SRS, and said acceptor groups are one or more of:

| | Donor | Donor |
|---|---|---|
| | Wavelength | Speed |
| Donor Group | Parameter | Parameter |
| H— H₃C— | Weak | Light |
| Alkyl—N═ | Strong | Light-Heavy |
| Aromatic— | Strong | Medium-Heavy |
| R—O— | Weak | Light-Heavy |
| R—S— | Weak | Medium-Heavy |
| 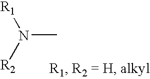 | Strong | Medium-Heavy |
| 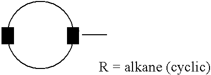 | Strong | Heavy |
| 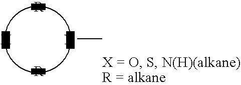 | Strong | Heavy |
| | Acceptor | |
|---|---|---|
| | Wavelength | Acceptor |
| Acceptor Group | Parameter | Speed Parameter |
| —F | Weak | Light |
| —NO₂ | Medium | Light |
| —CN | Weak | Light |
| —CF₃ | Weak | Medium |
| —SO₂CF₃ | Strong | Medium |
| —SO₂CH₃ | Strong | Medium |
| 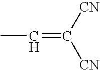 | Strong | Medium |
| 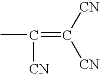 | Strong | Medium |
| 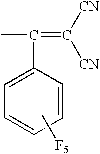 | Strong | Heavy, Branched |
| 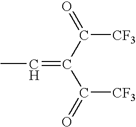 | Strong | Heavy, Branched |
| 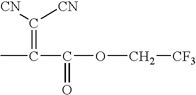 | Strong | Heavy, Branched |
| 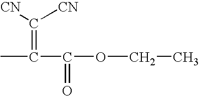 | Strong | Heavy, Branched |
| | Small Ring |
|---|---|
| Small Ring Substrate | Substrate Parameter |
| 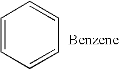 | Medium |
| 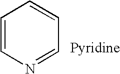 | Medium |
| 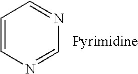 | Medium |
| 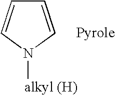 | Medium-Heavy |
| 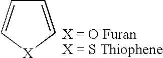 | Medium |
| 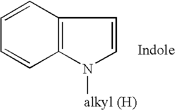 | Medium-Heavy |
| 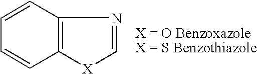 | Medium |
| 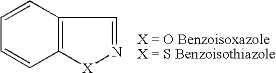 | Medium |
| 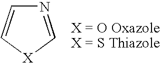 | Medium |
| 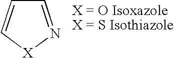 | Medium |
| 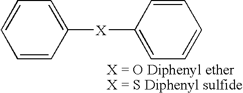 | Medium |
| 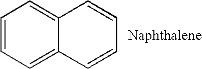 | Medium |
| 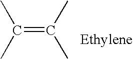 | Light |

4. The electrooptic chromophore of claim 1, which is dispersed in a host polymer.

5. The electrooptic chromophore of claim 4, wherein said host polymer is an acrylate polymer.

6. A mixture of at least two electrooptic chromophores of claim 1.

7. A mixture of at least one electrooptic chromophore of claim 1 and a conventional chromophore.

8. The electrooptic chromophore of claim 1, which is used in forming one or more of a Bragg grating modulator, a digital versatile disk (DVD), a laser printer, a Mach-Zender device, an optical wave guide, an optical attenuator, or an optical switch

9. The electrooptic chromophore of claim 1, which is formed into one or more of a coating or a slab.

10. An electrooptic chromophore for use in forming an electrooptic material (EO) comprising an electrooptic polymer and an electrooptic chromophore, which EO exhibits fast response, which comprises: 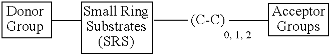
wherein the approximate size of the donor, SRS, and acceptor groups of the chromophore comprise:

| Donor Group | SRS | Acceptor Group |
|---|---|---|
| Heavy | Light | Light |
| Light or Medium | Light | Light, Medium, or |
| | | Branched Medium |
| Light or Medium | Light or Medium | Light or Medium |

11. The electrooptic chromophore of claim 10, which is one or more of: 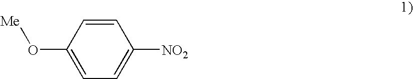
wherein for 404 nm operation said electrooptic chromophore is composed of a weak donor, medium SRS, and the measured absorption maximum is 302 nm, which makes said chromophore suitable for 405 nm operation; and
for fast response, the donor is light, the SRS is medium, and the acceptor is light; 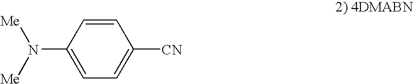
wherein for 405 nm operation, the molecule is composed of a strong donor, medium SRS, and weak acceptor and the measured absorption maximum is 290 nm, which makes said chromophore suitable for 405 nm operation; and
for fast response purposes, the donor is medium, the SRS is medium, and the acceptor is light; 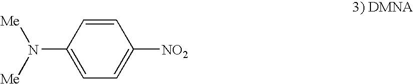
wherein 405 nm operation, the molecule is composed of a strong donor, medium SRS, and medium acceptor, and the measured absorption maximum of this chromophore is 407 nm, which precludes its use as an EO chromophore at 405 nm; and
for fast chromophore purposes, the donor is medium, the SRS is medium, and the acceptor is light; and 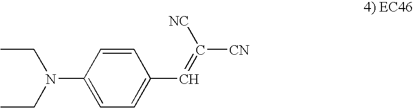
wherein for 405 nm operation, the molecule is composed of a strong donor, medium SRS, and medium acceptor wherein this molecule is not one of the acceptable combinations and the measured absorption maximum of this chromophore is 439 nm, which precludes its use as an EO chromophore at 405 nm; and
for fast chromophore purposes, the donor is medium, the SRS is medium, and the acceptor is medium;

12. The electrooptic chromophore of claim 10, which is dispersed in a host polymer.

13. The electrooptic chromophore of claim 10, wherein said host polymer is an acrylate polymer.

14. A mixture of at least two electrooptic chromophores of claim 10.

15. A mixture of at least one electrooptic chromophore of claim 10 and a conventional chromophore.

16. The electrooptic chromophore of claim 10, which is used in forming one or more of a Bragg grating modulator, a digital versatile disk (DVD), a laser printer, a Mach-Zender device, an optical wave guide, an optical attenuator, or an optical switch.

17. The electrooptic chromophore of claim 10, which is formed into one or more of a coating or a slab.

18. A polymerizable electrooptic chromophore, which comprises polymerizable acrylate esters of:

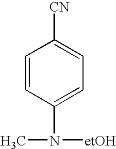

19. The polymerizable electrooptic chromophore of claim 18, which comprises:

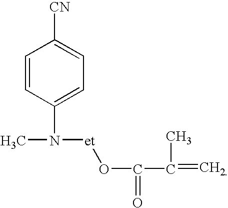

20. An electrooptic polymer, which comprises: 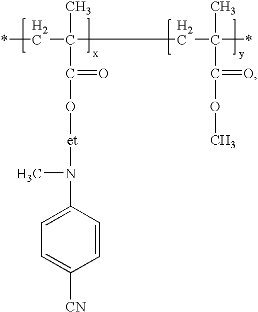
where, the mole fraction of x ranges from about 10 to 100 and the mole fraction of y ranges from 0 to about 90.

21. The electrooptic polymer of claim 19 blended with an acrylate polymer.
